(19) United States
(12) Patent Application Publication
(10) Pub. No.: US 2008/0293050 A1
(43) Pub. Date: Nov. 27, 2008

(54) Gene analysis for determination of a treatment characteristic

(75) Inventors: Edward S. Boyden, Cambridge, MA (US); Roderick A. Hyde, Redmond, WA (US); Muriel Y. Ishikawa, Livermore, CA (US); Edward K.Y. Jung, Bellevue, WA (US); Nathan P. Myhrvold, Medina, WA (US); Thomas A. Weaver, San Mateo, CA (US); Lowell L. Wood, JR., Bellevue, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/807,209

(22) Filed: May 25, 2007

(51) Int. Cl. C12Q 1/68; G01N 33/48

(52) U.S. Cl. 435/6; 702/20

(57) ABSTRACT

An apparatus, device, methods, computer program product, and systems are described that determine at least one amino acid sequence alteration of an amino acid sequence of a disease associated polypeptide, relative to an amino acid sequence of a corresponding non-disease associated polypeptide, identify a sub-sequence in the amino acid sequence of the disease associated polypeptide in which the amino acid sequence alteration occurs, relate the sub-sequence in the amino acid sequence of the disease associated polypeptide to a corresponding sub-sequence of the amino acid sequence of the corresponding non-disease associated polypeptide, and determine a treatment characteristic, based on the relating.

### TECHNICAL FIELD

This description relates to data handling techniques.

### SUMMARY

An embodiment provides a method. In one implementation, the method includes but is not limited to determining at least one amino acid sequence alteration of an amino acid sequence of a disease associated polypeptide, relative to an amino acid sequence of a corresponding non-disease associated polypeptide, identifying a sub-sequence in the amino acid sequence of the disease associated polypeptide in which the amino acid sequence alteration occurs, relating the sub-sequence in the amino acid sequence of the disease associated polypeptide to a corresponding sub-sequence of the amino acid sequence of the corresponding non-disease associated polypeptide, and determining a treatment characteristic, based on the relating. In addition to the foregoing, other method aspects are described in the claims, drawings, and text forming a part of the present disclosure.

An embodiment provides a computer program product. In one implementation, the computer program product includes but is not limited to a signal-bearing medium bearing one or more instructions for determining at least one amino acid sequence alteration of an amino acid sequence of a disease associated polypeptide, relative to an amino acid sequence of a corresponding non-disease associated polypeptide. The signal bearing medium also may bear one or more instructions for identifying a sub-sequence in the amino acid sequence of the disease associated polypeptide in which the amino acid sequence alteration occurs. The signal bearing medium also may bear one or more instructions for relating the sub-sequence in the amino acid sequence of the disease associated polypeptide to a corresponding sub-sequence of the amino acid sequence of the corresponding non-disease associated polypeptide. The signal bearing medium also may bear one or more instructions for determining a treatment characteristic, based on the relating. In addition to the foregoing, other computer program product aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

An embodiment provides a system, the system including a computing device including computer-executable instructions that when executed on the computing device, cause the computing device to determine at least one amino acid sequence alteration of an amino acid sequence of a disease associated polypeptide, relative to an amino acid sequence of a corresponding non-disease associated polypeptide, identify a sub-sequence in the amino acid sequence of the disease associated polypeptide in which the amino acid sequence alteration occurs, relate the sub-sequence in the amino acid sequence of the disease associated polypeptide to a corresponding sub-sequence of the amino acid sequence of the corresponding non-disease associated polypeptide, and determine a treatment characteristic, based on the relating. In addition to the foregoing, other system aspects are described in the claims, drawings, and text forming a part of the present disclosure.

An embodiment provides a treatment system, the treatment system comprising alteration determination logic configured to determine at least one amino acid sequence alteration of an amino acid sequence of a disease associated polypeptide, relative to an amino acid sequence of a corresponding non-disease associated polypeptide, alteration location logic configured to identify a sub-sequence in the amino acid sequence of the disease associated polypeptide in which the amino acid sequence alteration occurs, an alteration analyzer configured to relate the sub-sequence in the amino acid sequence of the disease associated polypeptide to a corresponding sub-sequence of the amino acid sequence of the corresponding non-disease associated polypeptide, and treatment logic configured to determine a treatment characteristic, based on the relating. In addition to the foregoing, other system aspects are described in the claims, drawings, and text forming a part of the present disclosure.

In addition to the foregoing, various other embodiments are set forth and described in the text (e.g., claims and/or detailed description) and/or drawings of the present description.

The foregoing is a summary and thus contains, by necessity, simplifications, generalizations and omissions of detail; consequently, those skilled in the art will appreciate that the summary is illustrative only and is not intended to be in any way limiting. Other aspects, features, and advantages of the devices and/or processes described herein, as defined by the claims, will become apparent in the detailed description set forth herein.

### BRIEF DESCRIPTION OF THE DRAWINGS

FIG. 1 illustrates an example clinical system in which embodiments may be implemented, perhaps in a device, to perform gene analysis for determination of a treatment characteristic.

FIG. 2 illustrates a conceptual graphical illustration of an amino acid sequencing and analysis that may be performed using the system of FIG. 1.

FIG. 3 illustrates an operational flow representing example operations related to gene analysis for determination of a treatment characteristic.

FIG. 4 illustrates an alternative embodiment of the example operational flow of FIG. 3.

FIG. 5 illustrates an alternative embodiment of the example operational flow of FIG. 3.

FIG. 6 illustrates an alternative embodiment of the example operational flow of FIG. 3.

FIG. 7 illustrates an alternative embodiment of the example operational flow of FIG. 3.

FIG. 8 illustrates an alternative embodiment of the example operational flow of FIG. 3.

FIG. 9 illustrates an alternative embodiment of the example operational flow of FIG. 3.

FIG. 10 illustrates an alternative embodiment of the example operational flow of FIG. 3.

FIG. 11 illustrates an alternative embodiment of the example operational flow of FIG. 3.

FIG. 12 illustrates an alternative embodiment of the example operational flow of FIG. 3.

FIG. 13 illustrates an alternative embodiment of the example operational flow of FIG. 3.

FIG. 14 illustrates an alternative embodiment of the example operational flow of FIG. 3.

FIG. 15 illustrates a partial view of an example computer program product that includes a computer program for executing a computer process on a computing device.

FIG. 16 illustrates an example system in which embodiments may be implemented.

The use of the same symbols in different drawings typically indicates similar or identical items.

### DETAILED DESCRIPTION

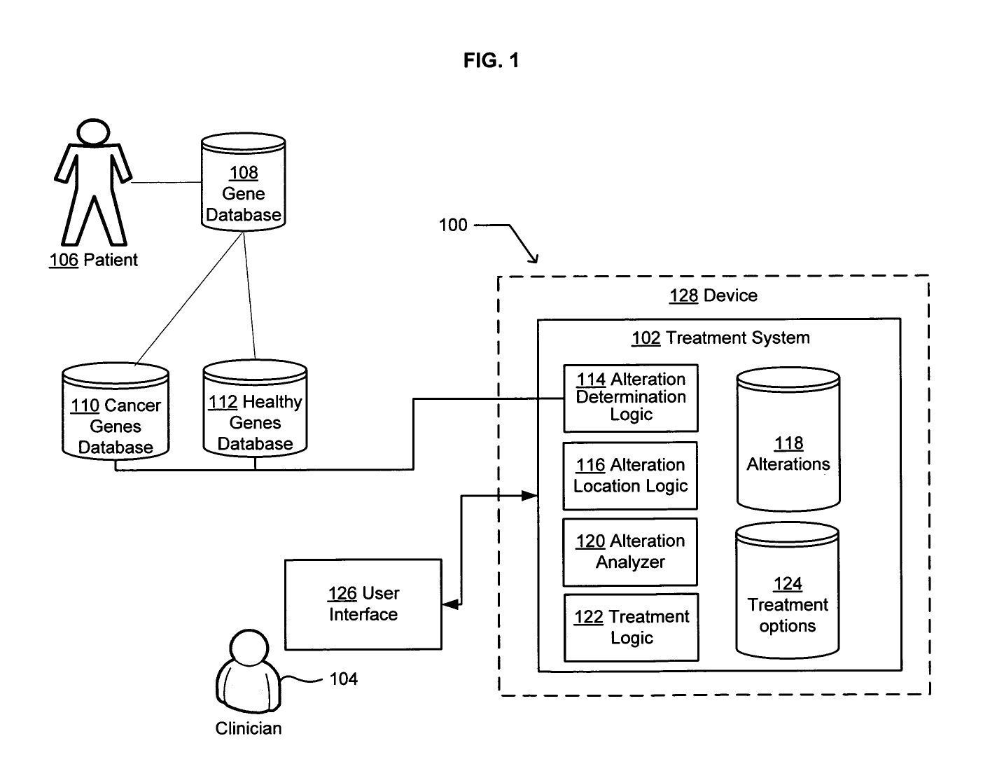

FIG. 1 illustrates an example clinical system 100 in which embodiments may be implemented, perhaps in a device, to perform gene analysis for determination of a treatment characteristic. The clinical system 100 includes a treatment system 102. The treatment system 102 may be used, for example, to determine a treatment characteristic that may be used in the treatment of one or more cancers or cancer-related illnesses. For example, the treatment system 102 may be used to determine individualized or highly-specialized cancer vaccines, based on a gene analysis of one or more cancer patients.

In FIG. 1, the treatment system 102 is used by a clinician 104. The clinician 104 may, for example, use the treatment system 102 to enter, store, request, process, or access clinical information such as, for example, the various examples provided herein. The clinician 104 may generally represent, for example, any person involved in health care, including, for example, a doctor, a nurse, a physician's assistant, or a medical researcher. The clinician 104 also may represent someone who is involved in health care in the sense of developing, managing, or implementing the treatment system 102, e.g., a software developer with clinical knowledge (or access to clinical knowledge), a database manager, or an information technologies specialist. Even more generally, some or all of various functions or aspects described herein with respect to the clinician 104 may be performed automatically, e.g., by an appropriately-designed and implemented computing device, or by software agents or other automated techniques.

A patient 106 generally represents any person with an illness, injury, or disease, or who is thought potentially to have such an illness, injury, or disease, or who may be wholly or partially healthy but who is nonetheless studied in order to determine information about such an illness, injury, or disease. The patient 106 also may represent or include other diagnostic and/or animal subjects that may be used in order, for example, to determine an efficacy of a particular medication or treatment, specific examples of which are provided herein. The patient 106 may represent a particular patient in a given clinical setting, such as in a doctor's office, or in a hospital, who is to be diagnosed and/or treated using the treatment system 102. The patient 106 also may represent the more abstract notion of a class of patients (e.g., patients having a certain age, gender, race, genetic makeup, or disposition to illness or disease), or, even more generally, may represent the general notion of a generic patient during basic research and/or development or application of various medical treatments or procedures. In the latter sense, the patient 106 also may represent a non-human animal (such as a primate) believed to be sufficiently similar to a human for the particular purposes that they may usefully substitute for such for the particular purposes.

In the example of FIG. 1, the patient 106 may represent one or more individuals suffering from cancer or cancer-related illnesses, such as, for example, breast cancer or colorectal cancer. Additionally, or alternatively, the patient 106 may represent an individual who is determined to be genetically predisposed to developing cancer, such as the cancers just mentioned.

A gene database 108 represents systems and/or devices for storage of genetic/genomic information for the patient 106. For example, the gene database 108 may store sequence information for Deoxyribonucleic acid (DNA), Ribonucleic Acid (RNA), messenger RNA (mRNA), nucleotides, bases, codons, amino acids, peptides, proteins, or virtually any other genetically-relevant information. As should be apparent from the above description, the gene database 108 may store such genetic information for an individual patient, and/or for a class or population of patients. As such, the gene database 108 also may store, or have access to, patient-relevant information, such as an identifier of the patient 106, or of a type of cancer experienced by the patient 106, or a location of a tumor(s) of the patient 106, or other information that may be used to store, access, classify, or otherwise utilize the genetic information in the gene database 108.

Further in FIG. 1, a cancer genes database 110 generally represents instances of the genetic information just referenced, or other genetic information, that are known or suspected to be associated with genetic information of a cancerous cell, tissue, organ, system, organ systems, or other bodily component. Similarly, a healthy genes database 112 may represent instances of the genetic information just referenced, or other genetic information, that are known or believed to be associated with genetic information of a healthy cell, tissue, organ, system, organ systems or other bodily component (in this context, the term healthy may be understood to be a relative term, e.g., non-cancerous, and does not necessarily imply freedom from any and all affliction or irregularity). Although in FIG. 1 the cancer genes database 110 and the healthy genes database 112 are illustrated for the sake of clarity and simplicity as separate elements, it will be appreciated that the gene database 108 may incorporate one or both of the cancer genes database 110 and/or the healthy genes database 112.

In FIG. 1, then, the gene database 108, the cancer genes database 110, and the healthy genes database 112 may be used to store gene sequence information for the patient 106. For example, such gene sequence information may include nucleotide sequences or amino acid sequences. The treatment system 102 may be configured to perform a comparative analysis between such sequence information stored in the cancer genes database 110 and the healthy genes database 112, and, based thereon, may be configured to deduce or determine, for example, treatment-relevant information for diagnosing, detecting, vaccinating for, or otherwise treating, a cancer of the patient 106. Further, the treatment system 102 may be configured to analyze such treatment-relevant information or other information, to determine a treatment characteristic to be associated with, or provided for the benefit of, the patient 106.

In this regard, it may be appreciated that cancers, cancerous tissue, tumors, carcinomas, or other malignancies or neoplasms may be considered to include, and/or may result from, mutations or other genetic alterations of genetic information in otherwise healthy cells within the body of the patient 106. Cancer may normally be associated with undesirable and largely unimpeded cell division of affected (cancerous) cells, and also may be associated, potentially, with a spread or metastasis of such cancerous cells throughout the body of the patient 106 (e.g., throughout a blood or lymphatic system of the patient 106). The uncontrolled division (and potentially metastasis) of cancer cells is generally associated with DNA damage that results in mutations or other alterations to genes encoding for the amino acids (which form proteins) that are in charge of cell division. Such alterations may be spontaneous, and/or may be caused by, accelerated by, or correlated with, known environmental or hereditary factors, including, e.g., certain chemicals, radioactive materials, or viruses.

As is known, it is possible that certain (e.g., mutated, cancerous, or otherwise altered) genetic material(s) may be used in the treatment of such cancers, e.g., to stimulate an immune response thereto. In this regard, for example, such altered genetic material (e.g., a sequence of amino acids forming a polypeptide) may be used as a vaccine against the cancer in question, or may be used against a spread of the cancer within the patient 106, or may otherwise be used to determine and use a treatment characteristic for treating the patient 106. As described in more detail below, the treatment system 102 may be used to determine and provide such treatment-relevant, immune-stimulating genetic materials in a number of ways, e.g., for use by the clinician 104 in treating the patient 106.

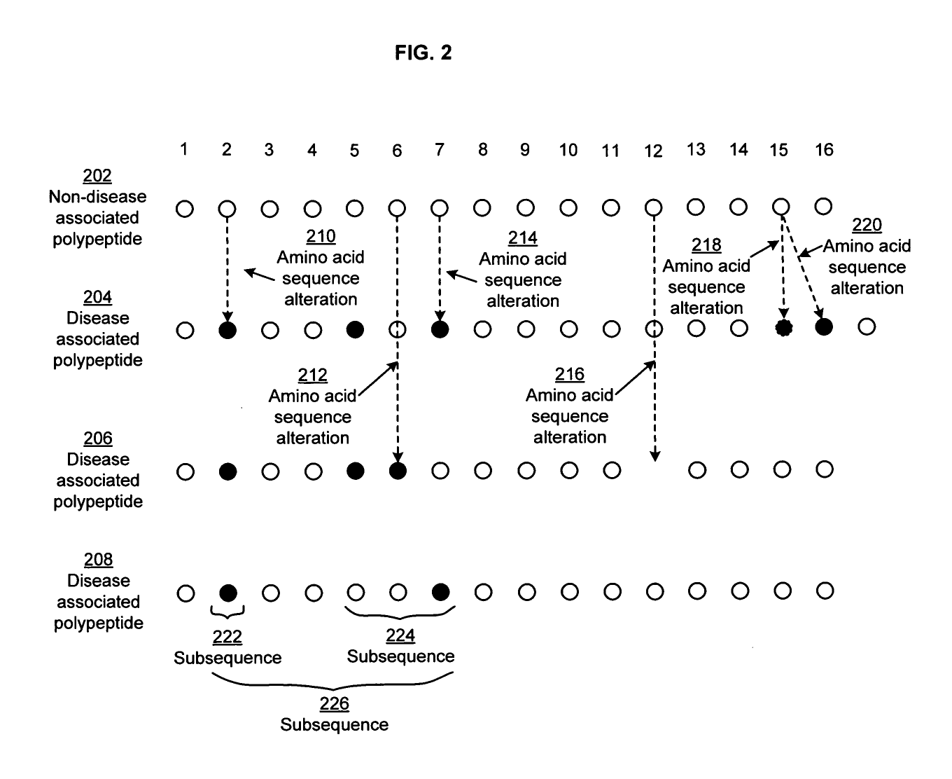

For example, as referenced above, and described in more detail below (e.g., as illustrated in FIG. 2), the treatment system 102 may be configured to perform a comparative analysis between mutated, or altered, genetic information (e.g., from the cancer genes database 110) and non-altered genetic information (e.g., from the healthy genes database 112). For example, the cancer genes database 110 may include amino acid sequence(s) within a disease associated polypeptide(s), while the healthy genes database 112 may include amino acid sequence(s) within a non-disease associated polypeptide(s) that are determined from healthy tissue of the patient 106.

In the example of FIG. 1, the treatment system 102 may include alteration determination logic 114 that may be configured to compare an amino acid sequence of a disease-associated polypeptide with an amino acid sequence of a non-disease associated polypeptide, as determined from the cancer genes database 110 and the healthy genes database 112, respectively. For example, the alteration determination logic 114 may include, or have access to, sequence alignment software that is operable to use probabilistic or statistical techniques to attempt to align such a disease associated amino acid sequence with a non-disease associated amino acid sequence. More specific examples of such sequence alignment techniques are provided herein, e.g., with respect to FIG. 2.

Based on a result(s) of such sequence alignment(s), alteration location logic 116 may be used to analyze the aligned sequences and determine where, within the sequences, an alteration (e.g., a mutation) may have occurred. The determined sequence alterations may be identified as being part of a sub-sequence (e.g., within a group or cluster of alterations, some or all of which may have a shared property or characteristic) within the disease associated amino acid sequence, e.g., using an alteration analyzer 120. Then, by relating the sub-sequence in such a disease associated amino acid sequence to a corresponding sub-sequence of the non-disease associated amino acid sequence, the alteration analyzer 120 may determine treatment-relevant information (e.g., information that may potentially be useful in diagnosing or otherwise treating the patient 106). For example, the alteration analyzer 120 may determine that a certain type of alteration, or alteration(s) having certain characteristics, may be strongly correlated with cancer in the patient 106, and may therefore be deduced, potentially, to provide a suitable vaccination agent for the cancer in question. The determined sequence alignments, alterations, sub-sequences, relations, and/or treatment-relevant information may be stored in whole or in part within an alterations database 118.

The alteration analyzer 120 may thus be configured to determine treatment-relevant information, and some, all, or none of the treatment-relevant information may be useful, either alone or in combination, in treating the patient 106. For example, treatment logic 122 may receive, or may otherwise access (e.g., from the alterations database 118), the treatment-relevant information from the alteration analyzer 120, which may include, for example, a plurality of polypeptide sequences that may be considered to be useful in stimulating an immune response of the patient 106 to invading cancer cells. The treatment logic 122 may thus be configured to determine which, if any, of these polypeptide sequences may be useful in treating the particular patient 106. For example, as described in more detail herein, the treatment logic 122 may be configured to select only such polypeptide sequences which are water-soluble (and thus more easily administrable to the patient 106), or may select (combinations of) such polypeptide sequences which are known not to stimulate an auto-immune response of the patient 106. In so doing, the treatment logic 122 may access treatment options from a treatment options database 124, which may specify, for example, various criteria to be used by the treatment logic 122 in utilizing the treatment-relevant information from the alterations database 118 to determine a treatment characteristic for treating the patient 106.

Thus, as described herein, the clinician 104 may interact with the treatment system 102, using a user interface 126. For example, the clinician 104 may use the user interface 126 to specify which of a plurality of patients (e.g., the patient 106) may require an operation of the treatment system 102, or to specify parameters for an operation of the treatment logic 122 (e.g., to request that the treatment logic 122 include a preference for selecting a treatment characteristic of a water-soluble polypeptide sequence over a non-autoimmune stimulating polypeptide sequence), to name just two examples of the use of the user interface 126. More generally, it will be appreciated that the user interface 126 allows the clinician 104 a convenient access to the treatment system 102 that may be used to determine a treatment characteristic, or to evaluate potential treatment characteristics, for the patient 106.

Also in FIG. 1, the treatment system 102 is illustrated as possibly being included within a device 128. The device 128 may include, for example, a mobile computing device, such as a personal digital assistant (PDA), or a laptop computer. Of course, virtually any other computing device may be used to implement the treatment system 102, such as, for example, a workstation, a desktop computer, or a tablet PC. Of course, in practice, not all of the treatment system 102 need be implemented on a single computing device. For example, the treatment logic 122 may be implemented in part on a first device that is used locally by the clinician 104, while the alteration determination logic 114, the alteration location logic 116, and the alteration analyzer 120 may be stored and executed on a remote, networked device(s). In this way, the clinician 104, who may be operating in the field, e.g., in an office and/or hospital environment, may be relieved of a responsibility to update, manage, or manipulate the contents of the alterations database 118, and may focus on accessing the data therein, using the treatment logic 122 and data in the treatment options database 124, to determine/evaluate (possible) treatment characteristics for the patient 106.

FIG. 2 illustrates a conceptual graphical illustration of an amino acid sequencing and analysis that may be performed using the system 100 of FIG. 1. FIG. 2 provides a conceptual illustration of amino acid sequences forming polypeptide sequences that may be used by, or produced by, the treatment system 102. FIG. 2 is not, therefore, intended to provide a complete, detailed, or comprehensive description or definition of types or characteristics of amino acid sequences, and/or alterations thereof.

Rather, FIG. 2 merely illustrates that a non-disease associated polypeptide 202 may include a sequence of amino acids that are illustrated at sites/positions labeled 1 through 16, as shown. The representation of the sequence of amino acids within the non-disease associated polypeptide 202 is not intended to have a particular biological significance (e.g., does not represent any particular amino acid sequence(s) or site(s)), but rather just indicates that virtually any sequence of amino acids may be designated as having a position within the overall polymer/molecular chain, so that, as described herein, such sequences from different cells, and/or from different patients, may be aligned with one another for comparison thereof.

For example, the non-disease associated polypeptide 202 may be taken from a healthy cell/sample of the patient 106, or may represent one or more samples of non-disease associated sequences from the population at large, or from a particular class/group (e.g., a group sharing an ethnicity, gender, age range, or other characteristic). As such, the non-disease associated polypeptide 202 may be stored in the healthy gene database 112.

The non-disease associated polypeptide 202 may thus serve as a baseline, or point of comparison, for one or more disease associated polypeptides, e.g., disease associated polypeptides 204, 206, 208. For example, each of the disease associated polypeptides 204, 206, 208 may be taken from the patient 106, e.g., may be taken from three sites within a particular person/patient, or may be taken from three different persons/patients, each of whom may be suffering from a certain type of cancer, e.g., breast cancer or colon cancer. The disease associated polypeptides 204, 206, 208 may be stored in, and accessed from, the cancer genes database 110.

As illustrated in FIG. 2, alignment of the disease associated polypeptides 204, 206, 208 with the non-disease associated polypeptide 202 may allow for the observation of amino acid sequence alterations 210-220, e.g., mutations that may occur in healthy cells that may cause, or be correlated with, cancer or cancer-related illnesses. As referenced above, sequence alignment software exists that may be implemented by, or in conjunction with, the alteration determination logic 114 of FIG. 1. Such sequence alignment software may implement one or more of, for example, the Smith-Waterman algorithm, the Needleman-Wunsch algorithm, and/or the basic local alignment search tool (BLAST) software. Such algorithms and software are known, for example, to compare a first sequence of amino acids (or other sequences, such as nucleotides of DNA sequences) with one or more comparison sequences, so as to determine sequences sharing a certain level or type of similarity. Such alignment tools may be used, for example, to identify similar sequences, such as when a gene sequence of a mouse (or other test animal) is compared against the human genome to determine whether a similar/corresponding gene may exist in humans.

In the present context, however, the alteration determination logic 114 may be used to compare each of the disease associated polypeptides 204, 206, 208 with the non-disease associated polypeptide 202, in order to be able to observe or otherwise determine the amino acid sequence alterations 210-220. Examples of the types and nature of such alterations are well-known, but, generally speaking, such alterations may include point/substitution mutations (in which a first amino acid is replaced with a second amino acid, e.g., tryptophan replaced by lycene), an insertion (in which an extraneous amino acid is added to a sequence), a deletion (in which an amino acid is removed from a sequence), or a shift (in which an amino acid moves from a first site/position to a second site/position within a sequence). Alterations may include changes to a physical property of the amino acid (e.g., size), or a chemical property (e.g., acidity). Of course, various combinations of these alterations, and other alterations, may occur, as well. Additional, non-exhaustive examples of amino acid sequence alterations are provided, below.

FIG. 2 illustrates some examples of such alterations. In general, in FIG. 2, amino acid sequence alterations are designated by filled circles (or, in the case of deletions, missing circles). For example, the amino acid sequence alterations 210-214 represent a point mutation(s), in which an amino acid at sites 2, 6, and 7, respectively, are mutated into different amino acids, or into amino acids having a different property (e.g., a different chemical or physical property), at the same sites 2, 6, and 7. As shown, the amino acid sequence alterations 210 and 214 are observed to occur between the non-disease associated polypeptide 202 and the disease associated polypeptide 204, while the amino acid sequence alteration 212 occurs between the non-disease associated polypeptide 202 and the disease associated polypeptide 206.

The amino acid sequence alteration 216 represents a deletion, in which an amino acid at site 12 in the non-disease associated polypeptide 202 is deleted (not present) in the disease associated polypeptide 206. Meanwhile, the amino acid sequence alteration 218 may represent an insertion at site 15, perhaps in conjunction with a shift of the previous amino acid to the site 16, as illustrated in FIG. 2 as the amino acid sequence alteration 220.

As referenced above, the amino acid sequence alterations 210-220, and others not specifically labeled, may be correlated, to varying extents, with cancer or cancer-related illnesses in patients. By analyzing such amino acid sequence alterations, as explained herein, the treatment system 102 may be operable to determine which, if any, are potential epitopes (e.g., vaccination agents) that may be used to detect, mitigate, or eliminate disease in the patient 106.

For example, once the just-described alignment and alteration detection have taken place, the alteration location logic 116 may be used to identify sub-sequences of the amino acid sequences of the various polypeptides 202-208. For example, having determined that a number of alterations exist, there may exist a number of possibilities as to how to identify or classify these alterations, e.g., relative to the non-disease associated polypeptide, or relative to one another. Such identifications/classifications may be useful, as described herein, in determining whether and how the identified sub-sequences may be useful in treating the patient 106.

For example, in FIG. 2, a sub-sequence 222 is illustrated that includes the (single) amino acid at site 2 of each of the disease associated polypeptides 204, 206, 208. For example, the disease associated polypeptides 204, 206, 208 may be taken from three patients having colon cancer, so that determining the same or similar alteration at the same site within all three (or more) of the patients may indicate value of the sub-sequence 222 in treating the three patients, or other patients. It will be appreciated that the sub-sequence 222 may represent a case where a first amino acid at site 2 of the non-disease associated polypeptide 202 is transformed into a second amino acid at site 2 of all of the disease associated polypeptides 204, 206, 208. In other examples, the sub-sequence 222 may represent a case where a first amino acid at site 2 of the non-disease associated polypeptide 202 is transformed into one of a number of possible amino acids at site 2 of all of the disease associated polypeptides 204, 206, 208, where all of the number of possible amino acids share some common characteristic(s) (e.g., polarity, acidity, or other chemical or physical property).

Similarly in FIG. 2, a sub-sequence 224 may be identified that includes a group or cluster of amino acids at sites 5, 6, and 7. As illustrated, all three of the disease associated polypeptides 204, 206, 208 share at least one amino acid sequence alteration within the sub-sequence 224. Thus, again, the sub-sequence 224 may be of use in treating some or all of the patients from whom the disease associated polypeptides 204, 206, 208 were taken, or other patients.

As described in more detail herein, there may be a number of different techniques or criteria used by the alteration location logic 116 in identifying sub-sequences of the disease associated polypeptides 204, 206, 208. Consequently, results may vary as to which such sub-sequences are identified. For example, although the sub-sequences 222 and 224 are illustrated in FIG. 2, it may be appreciated that a potentially equally-valid identification may include identifying a sub-sequence 226 that includes amino acids at the sites 2-7. Different techniques and criteria for identifying sub-sequences of the disease associated polypeptides 204, 206, 208 are provided in more detail, below.

Once identified, such sub-sequences may be related to corresponding sub-sequences of the non-disease associated polypeptide 202, in order to determine whether, how, and to what extent the identified sub-sequences 222, 224, and/or 226 of the disease associated polypeptides 204, 206, 208 may be useful in treating the patient 106. For example, the alteration analyzer 120 may be used to analyze the sub-sequences in this manner, as described in more detail, herein.

Thus, the alteration analyzer 120 may provide treatment relevant information, including, for example, the sub-sequences 222, 224, 226 of the disease associated polypeptides 204, 206, 208, as well as characteristics of such sub-sequences, e.g., relative to the non-disease associated polypeptide 202. Then, such information, and related information, may be stored in the alterations database 118, and the treatment logic 122 may access the alterations database 118 and the treatment options database 124, as described in more detail herein, in order to determine whether and how to select and apply desired portions thereof in determining a treatment characteristic for treating the patient 106.

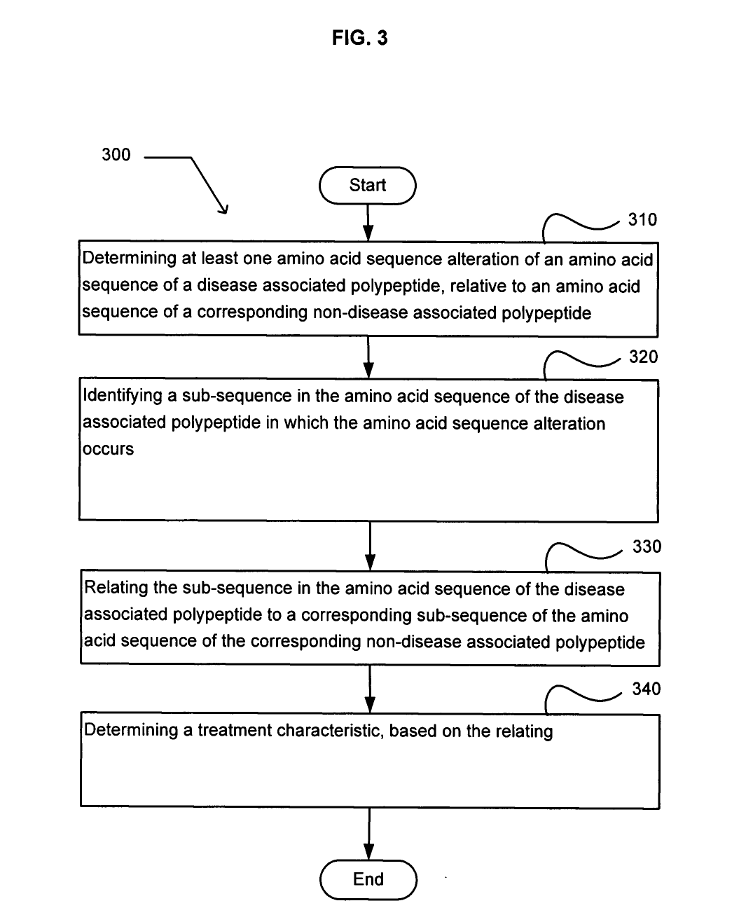

FIG. 3 illustrates an operational flow 300 representing example operations related to gene analysis for determination of a treatment characteristic. In FIG. 3 and in following figures that include various examples of operational flows, discussion and explanation may be provided with respect to the above-described examples of FIGS. 1 and 2, and/or with respect to other examples and contexts. However, it should be understood that the operational flows may be executed in a number of other environments and contexts, and/or in modified versions of FIG. 1. Also, although the various operational flows are presented in the sequence(s) illustrated, it should be understood that the various operations may be performed in other orders than those which are illustrated, or may be performed concurrently.

After a start operation, the operational flow 300 moves to a determining operation 310, where at least one amino acid sequence alteration of an amino acid sequence of a disease associated polypeptide may be determined, relative to an amino acid sequence of a corresponding non-disease associated polypeptide. For example, the alteration determination logic 114 may be configured to obtain the non-disease associated polypeptide 202 from the healthy genes database 112, as well as the disease-associated polypeptide 204 from the cancer genes database 110. The alteration determination logic 114 may be configured to align the disease-associated polypeptide 204 with the non-disease associated polypeptide 202, and then determine at least the amino acid sequence alteration(s) 210, 214, 218 and/or 220. Such process(es) may be repeated for the disease associated polypeptide sequences 206, 208, and/or other disease associated polypeptide sequences that also may be obtained, e.g., from the cancer genes database 110.

In operation 320, a sub-sequence in the amino acid sequence of the disease associated polypeptide may be identified in which the amino acid sequence alteration occurs. For example, the alteration location logic 116 may define the sub-sequence(s) 222, 224, and/or 226 in one or more of the disease associated polypeptides 204, 206, 208. As referenced above, it will be appreciated that it may be difficult simply to line up amino acid sequences, side-by-side, for alignment, since it may be difficult to determine when a particular altered sequence ends and the unaltered sequence begins (e.g., due to duplications, deletions, and other alterations). Rather, as described in more detail herein, probabilistic or statistical methods may be used to determine whether and where such alterations occur, and to identify sub-sequences accordingly.

In operation 330, the sub-sequence in the amino acid sequence of the disease associated polypeptide may be related to a corresponding sub-sequence of the amino acid sequence of the corresponding non-disease associated polypeptide. For example, in a straight-forward example, the alteration analyzer 120 may determine that the sub-sequence in the amino acid sequence of the disease associated polypeptide 204 corresponds directly to a particular sub-sequence in the amino acid sequence of the non-disease associated polypeptide 202, such as in the amino acid sequence alteration 210, which causes the alteration at site 2 within the disease associated polypeptide 204 relative to the corresponding amino acid at site 2 within the non-disease associated polypeptide 202 (e.g., within the sub-sequence 222). Similar comments apply to the sub-sequences 224 and 226. In general, it will be appreciated that the term corresponding may include, for example, any correspondence that is relevant for the purpose of determining a treatment characteristic, and does not necessarily imply the site-to-site correspondence illustrated in the sub-sequence 222 of FIG. 2. For example, relating the identified sub-sequence in the amino acid sequence of the disease associated polypeptide to a corresponding sub-sequence in the amino acid sequence of the non-disease associated polypeptide may include determining that there is no one-to-one correspondence between the two sub-sequences, such as when an entirely new amino acid(s) is inserted into the disease-associated polypeptide. In fact, as described in more detail herein, an extent of correspondence between the sub-sequences (or lack thereof) may be a useful indicator, since, for example, alterations that have a low correspondence to the sub-sequence in the amino acid sequence of the non-disease associated polypeptide may be relatively more useful as a vaccine, e.g., may be less likely to stimulate an auto-immune response in the patient 106.

In operation 340, a treatment characteristic may be determined, based on the relating. For example, the treatment logic 122 may be configured to access information from the alterations analyzer 120 (e.g., from the alterations database 118) and from the treatment options database 124, and may determine a treatment characteristic based thereon. Although a number of examples of the nature and operation of the treatment logic 122 are provided herein, it will generally be appreciated that the treatment characteristic may include, for example, any information that may be useful in treating the patient 106, where the treatment may include, for example, diagnosis, vaccination, therapy, immunotherapy, palliative care, or curative measures, as well as any agent (e.g., a medicine, delivery agent, or other substance) or data (e.g., patient or disease-relevant data) that may be associated therewith. As described herein, the treatment logic 122 may be configured to determine these and other treatment characteristics, for example, for a single person or for large groups of patients. In the former case, for example, all of the non-disease associated polypeptide 202 and the disease associated polypeptides 204, 206, 28 may be taken from a single person, so that a resulting vaccine or other treatment agent may be designed in a manner that is highly specific and individualized with respect to the person.

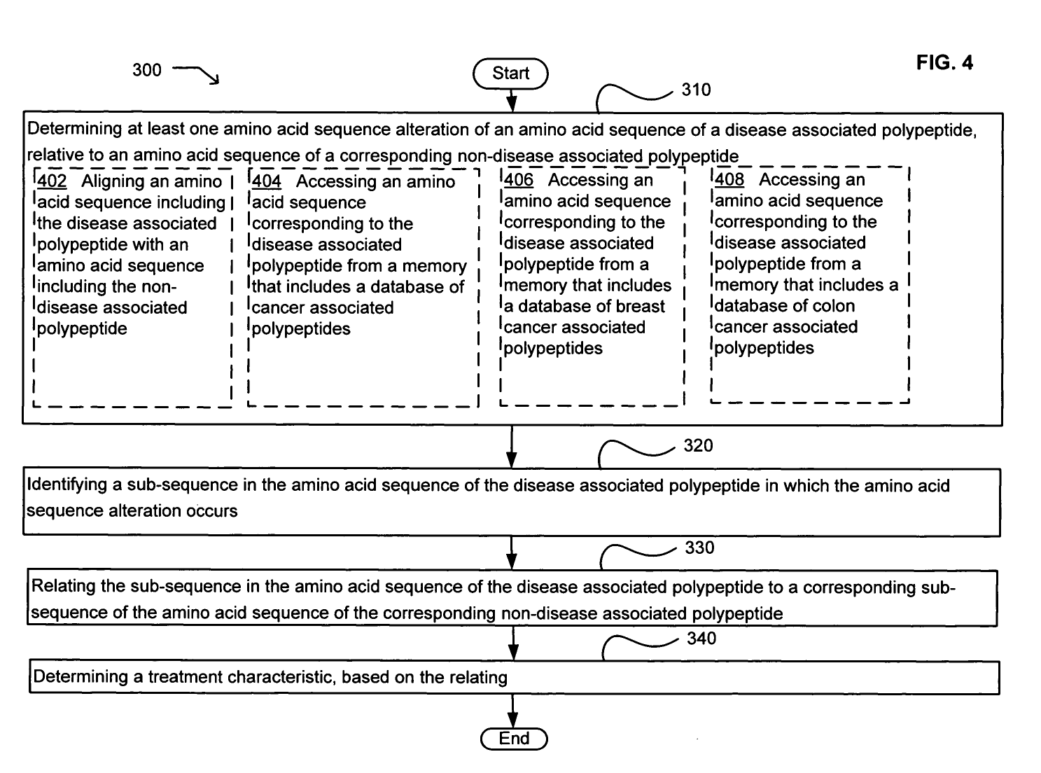

FIG. 4 illustrates alternative embodiments of the example operational flow 300 of FIG. 3. FIG. 4 illustrates example embodiments where the determining operation 310 may include at least one additional operation. Additional operations may include an operation 402, an operation 404, an operation 406, and/or an operation 408.

At the operation 402, an amino acid sequence including the disease associated polypeptide may be aligned with an amino acid sequence including the non-disease associated polypeptide. For example, the alteration determination logic 114 may be configured to align an amino acid sequence including the disease associated polypeptide 204 with an amino acid sequence including the non-disease associated polypeptide 202.

At the operation 404, an amino acid sequence corresponding to the disease associated polypeptide may be accessed from a memory that includes a database of cancer associated polypeptides. For example, the alteration determination logic 114 may be configured to access an amino acid sequence corresponding to the disease associated polypeptide 204 from the cancer genes database 110.

At the operation 406, an amino acid sequence corresponding to the disease associated polypeptide may be accessed from a memory that includes a database of breast cancer associated polypeptides. For example, the alteration determination logic 114 may be configured to access an amino acid sequence corresponding to the disease associated polypeptide 204 from the cancer genes database 110, where the cancer genes database 110 may be used to store genetic information related to the patient 106, who may represent a breast cancer patient(s).

At the operation 408, an amino acid sequence corresponding to the disease associated polypeptide may be accessed from a memory that includes a database of colon cancer associated polypeptides. For example, the alteration determination logic 114 may be configured to access an amino acid sequence corresponding to the disease associated polypeptide 204 from the cancer genes database 110, where the cancer genes database 110 may be used to store genetic information related to the patient 106, who may represent a colon cancer patient(s).

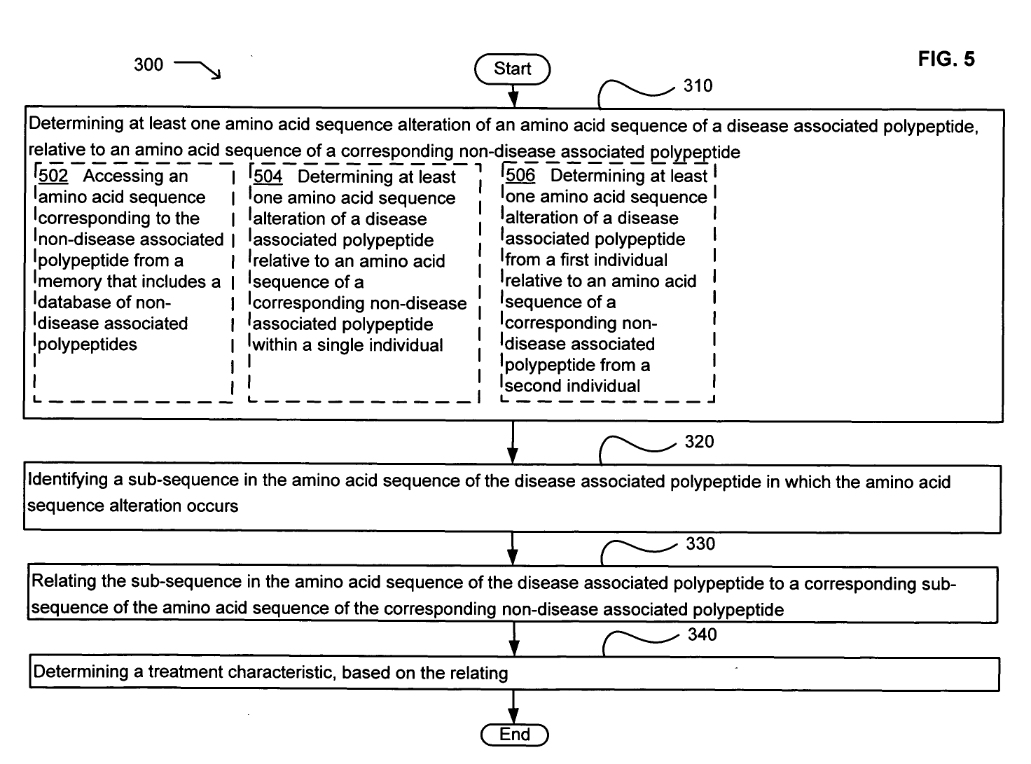

FIG. 5 illustrates alternative embodiments of the example operational flow 300 of FIG. 3. FIG. 5 illustrates example embodiments where the determining operation 310 may include at least one additional operation. Additional operations may include an operation 502, an operation 504, and/or an operation 506.

At the operation 502, an amino acid sequence corresponding to the non-disease associated polypeptide may be accessed from a memory that includes a database of non-disease associated polypeptides. For example, the alteration determination logic 114 may be configured to access an amino acid sequence corresponding to the non-disease associated polypeptide 202 from the healthy genes database 112, where the healthy genes database 112 may be used to store genetic information related to the patient 106 and taken from non-cancerous portions of the body of the patient 106.

At the operation 504, at least one amino acid sequence alteration of a disease associated polypeptide may be determined relative to an amino acid sequence of a corresponding non-disease associated polypeptide, within a single individual. For example, the alteration determination logic 114 may be configured to access an amino acid sequence corresponding to the non-disease associated polypeptide 202 from the healthy genes database 112, and may be configured to access an amino acid sequence corresponding to the disease associated polypeptide 204 from the cancer genes database 110, where the respective polypeptides 202, 204 may be associated with the (single) patient 106 within the respective databases 112, 110. Thus, as described in more detail herein, the treatment system 102 may be configured to determine a treatment characteristic that is highly specialized and individualized to the single patient 106.

At the operation 506, at least one amino acid sequence alteration of a disease associated polypeptide from a first individual may be determined relative to an amino acid sequence of a corresponding non-disease associated polypeptide from a second individual. For example, the alteration determination logic 114 may be configured to access an amino acid sequence corresponding to the non-disease associated polypeptide 202 from the healthy genes database 112 and marked therein as being associated with at least one other individual besides the patient 106, and may be configured to access an amino acid sequence corresponding to the disease associated polypeptide 204 from the cancer genes database 110 and marked therein as being associated with the patient 106. Thus, as described in more detail herein, the treatment system 102 may be configured to determine a treatment characteristic for the patient 106, using data associated with a larger population, and thereby providing a more varied source of comparison for relating the disease associated polypeptide 204 to the non-disease associated polypeptide 202. For example, the at least one other individual may be a member of a population of which the patient 106 is also a member (for example, characterized by age, gender, ethnicity, genetic pre-disposition, or other characteristic). In other example implementations, the at least one other individual may be a member of a randomly-selected population of individuals.

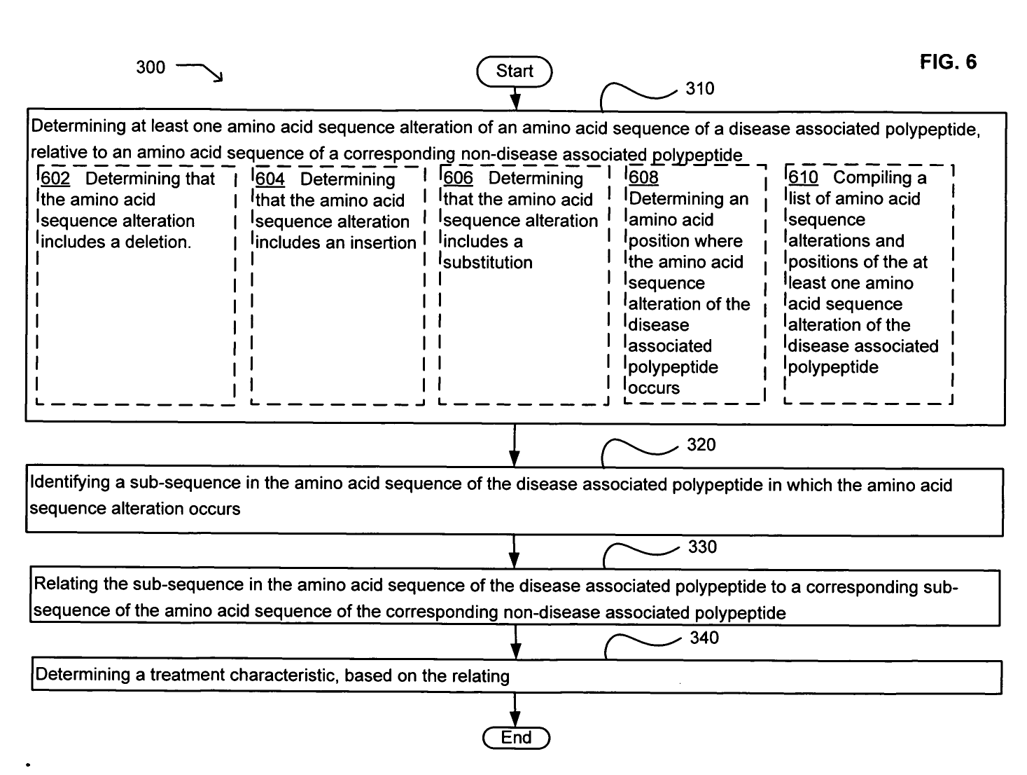

FIG. 6 illustrates alternative embodiments of the example operational flow 300 of FIG. 3. FIG. 6 illustrates example embodiments where the determining operation 310 may include at least one additional operation. Additional operations may include an operation 602, an operation 604, an operation 606, an operation 608, and/or an operation 610.

At the operation 602, it may be determined that the amino acid sequence alteration includes a deletion. For example, the alteration determination logic 114 may be configured to determine the amino acid sequence alteration 216 of FIG. 2, in which an amino acid at site 12 of the non-disease associated polypeptide 202 is deleted from the corresponding site in the disease associated polypeptide 206.

At the operation 604, it may be determined that the amino acid sequence alteration includes an insertion. For example, the alteration determination logic 114 may be configured to determine the amino acid sequence alteration 218 of FIG. 2, in which an amino acid is inserted at site 15 of the disease associated polypeptide 204.

At the operation 606, it may be determined that the amino acid sequence alteration includes a substitution. For example, the alteration determination logic 114 may be configured to determine the amino acid sequence alteration 210 of FIG. 2, in which an amino acid at site 2 of the non-disease associated polypeptide 202 is substituted for at the corresponding site in the disease associated polypeptide 204.

At the operation 608, an amino acid position where the amino acid sequence alteration of the disease associated polypeptide occurs may be determined. For example, the alteration determination logic 114 may be configured to determine positions of the amino acid sequence alterations 210-220 of FIG. 2. For example, the alteration determination logic may be configured to determine that the amino acid sequence alteration 220 includes a shift of an amino acid from site 15 to site 16, as opposed to a substitution at site(s) 16.

At the operation 610, a list of amino acid sequence alterations and positions of the at least one amino acid sequence alteration of the disease associated polypeptide may be compiled. For example, the alteration determination logic 114 may be configured to compile a list of amino acid sequence alterations and positions of the at least one amino acid sequence alteration of one or more of the disease associated polypeptides 204, 206, 208, e.g., for storage in the alterations database 118 (and later access therefrom by the alteration location logic 116 for identification of one or more sub-sequences, as described in more detail, herein).

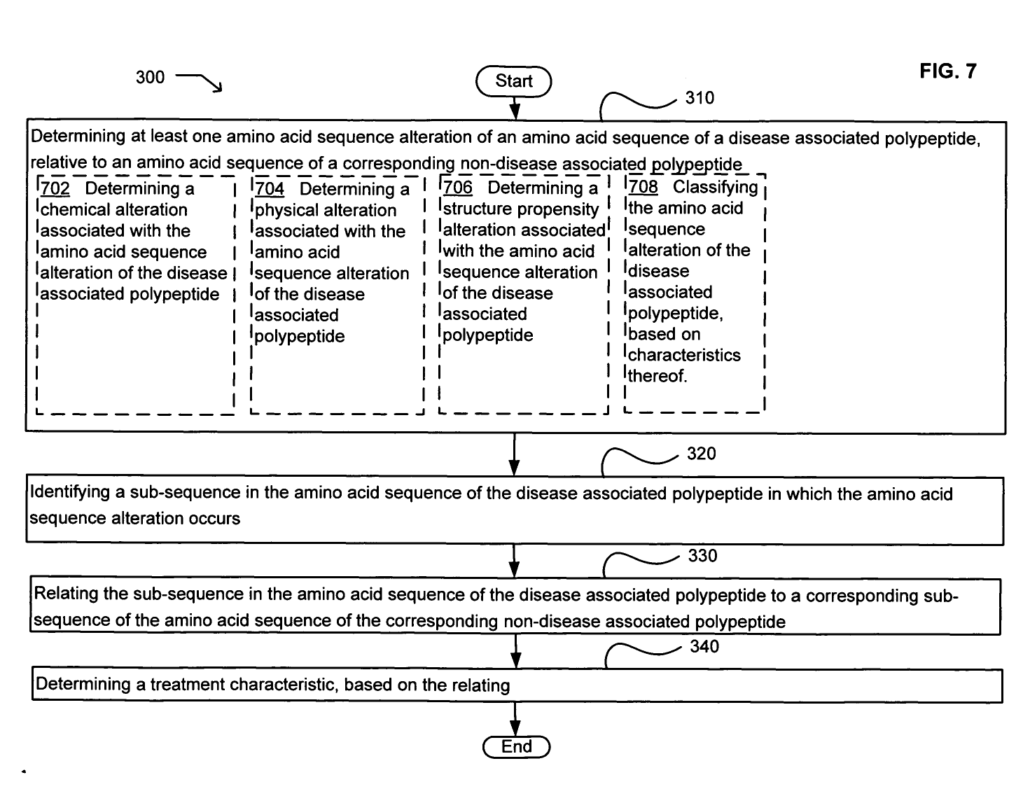

FIG. 7 illustrates alternative embodiments of the example operational flow 300 of FIG. 3. FIG. 7 illustrates example embodiments where the determining operation 310 may include at least one additional operation. Additional operations may include an operation 702, an operation 704, an operation 706, and/or an operation 708.

At the operation 702, a chemical alteration associated with the amino acid sequence alteration of the disease associated polypeptide may be determined. For example, the alteration determination logic 114 may be configured to determine a change in acidity associated with the amino acid sequence alteration 210.

At the operation 704, a physical alteration associated with the amino acid sequence alteration of the disease associated polypeptide may be determined. For example, the alteration determination logic 114 may be configured to determine a change in size associated with the amino acid sequence alteration 214.

At the operation 706, a structure propensity alteration associated with the amino acid sequence alteration of the disease associated polypeptide may be determined. For example, the alteration determination logic 114 may be configured to determine a change in structure propensity associated with amino acid sequence alterations within the sub-sequence 224. In this regard, it will be appreciated that linear amino acid sequences may have different propensities to assume different structural, three-dimensional shapes (e.g., “folds”) that are associated with one or more biological functions, and that amino acid sequence alterations may affect these propensities, thereby affecting, potentially, the corresponding biological functions.

At the operation 708, the amino acid sequence alteration of the disease associated polypeptide may be classified, based on characteristics thereof. For example, the alteration determination logic 114 may be configured to classify any of the amino acid sequence alterations 210-220 as one or more of the above categories (e.g., chemical, physical, or structural), or another category. As described in more detail herein, such classifications may represent examples of treatment-relevant information that may be used by the treatment logic 122 to determine a treatment characteristic for the patient 106.

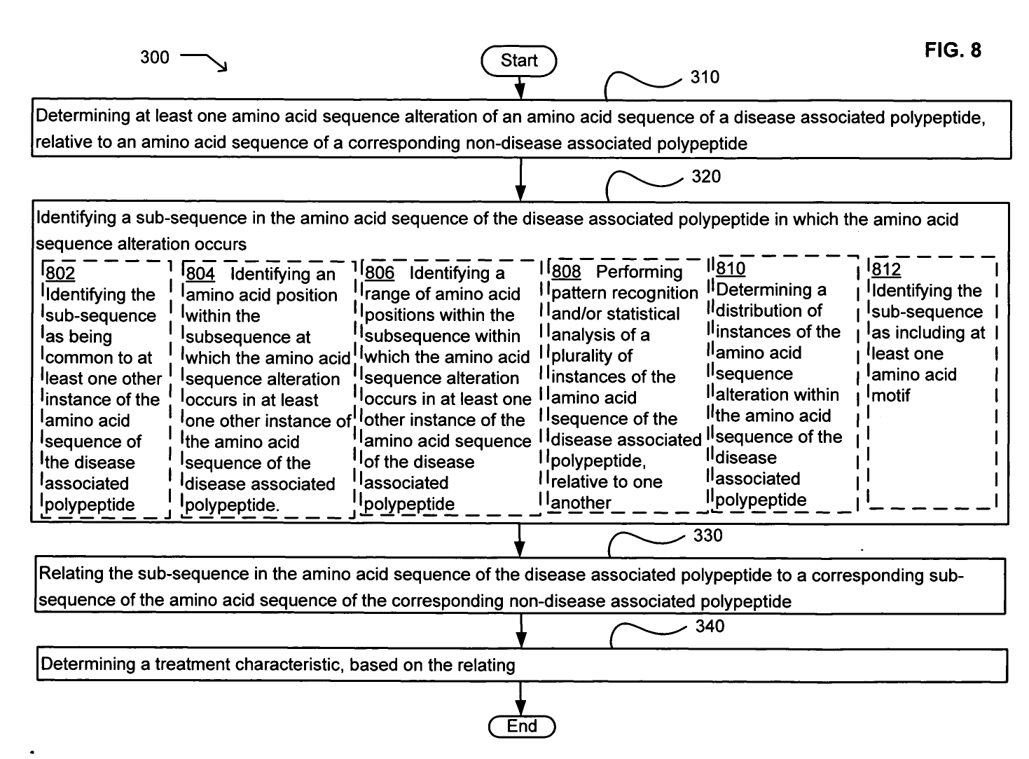

FIG. 8 illustrates alternative embodiments of the example operational flow 300 of FIG. 3. FIG. 8 illustrates example embodiments where the identifying operation 320 may include at least one additional operation. Additional operations may include an operation 802, an operation 804, an operation 806, an operation 808, and operation 810, and/or an operation 812.

At the operation 802, the sub-sequence may be identified as being common to at least one other instance of the amino acid sequence of the disease associated polypeptide. For example, the alteration location analyzer 116 may be configured to determine that the amino acid sequence alteration 210 may be identified as the sub-sequence 222, and may determine that the sub-sequence 222 (e.g., the amino acid sequence alteration 210) may be common to the disease associated polypeptides 206, 208, as well. In other words, the fact that the same substitution alteration occurs between the non-disease associated polypeptide 202 and all three instances of the disease associated polypeptides 204, 206, 208 may be recognized by the alteration location logic 116 and used to identify the sub-sequence 222.

At the operation 804, an amino acid position within the sub-sequence at which the amino acid sequence alteration occurs may be identified in at least one other instance of the amino acid sequence of the disease associated polypeptide. For example, and continuing the example just given, the alteration location analyzer 116 may be configured to recognize that all of the recognized alterations occur at site 2 of the polypeptides 202-208.

At the operation 806, a range of amino acid positions within the sub-sequence within which the amino acid sequence alteration occurs may be identified in at least one other instance of the amino acid sequence of the disease associated polypeptide. For example, the alteration location analyzer 116 may be configured to recognize that amino acid sequence alterations occur within a range of sites 5-7 (e.g., the amino acid sequence alterations 212 and 214). That is, although it is apparent from FIG. 2 that the illustrated alterations are not identically-positioned within the disease associated polypeptides 204, 206, 208, it may be observed that at least one alteration occurs within the range of sites 5-7 for each instance of the disease associated polypeptides 204, 206, 208. Consequently, the alteration location logic 116 may identify the sub-sequence 224 as such. Similar comments apply to the range of sites 2-7, which, as described herein, may be identified as the sub-sequence 226. For the sake of contrast, it may be observed that no alterations occur at any of sites 8-11 in any of the disease-associated polypeptides 204, 206, 208, so that the alteration location logic 116 may be unlikely to identify a sub-sequence within this portion of the disease associated polypeptides 204, 206, 208.

At the operation 808, pattern recognition and/or statistical analysis of a plurality of instances of the amino acid sequence of the disease associated polypeptide, relative to one another, may be performed. For example, the alteration location analyzer 116 may be configured to perform pattern recognition to identify the patterns just mentioned, e.g., the pattern of at least one amino acid sequence alteration within the sites 5-7 for all of the disease associated polypeptides 204, 206, 208. The pattern recognition and/or statistical analysis may involve determination(s) of the types, classifications, or interactions of the recognized alterations, or of the positions themselves. For example, the alteration location analyzer 116 may determine that certain (combinations of) sites and/or alterations may be associated with a catalytic nature of the relevant amino acids, so that a biological requirement or expectation for a speed of a chemical reaction may not be met, or may be exceeded. Thus, the alteration location analyzer 116 may identify such sites/alterations as a relevant sub-sequence, since the disturbance of such chemical reactions may be relevant to a presence or severity of cancer in the patient 106.

At the operation 810, a distribution of instances of the amino acid sequence alteration within the amino acid sequence of the disease associated polypeptide may be determined. For example, the alteration location analyzer 116 may be configured to determine a concentration of amino acid sequence alterations, or, conversely, may be configured to determine a relatively smooth spread or distribution of such alterations, where such distributions may have greater or lesser correlation with cancer in the patient 106.

At the operation 812, the sub-sequence may be identified as including at least one amino acid motif. For example, the alteration location analyzer 116 may be configured to identify a sub-sequence of amino acids that meet a known definition of a sequence motif (e.g., defined as “amino acid A; followed by any of amino acids A, B, or C; followed by any amino acid other than D or E”), which may be associated with a known biological function (e.g., catalytic activity, as just mentioned).

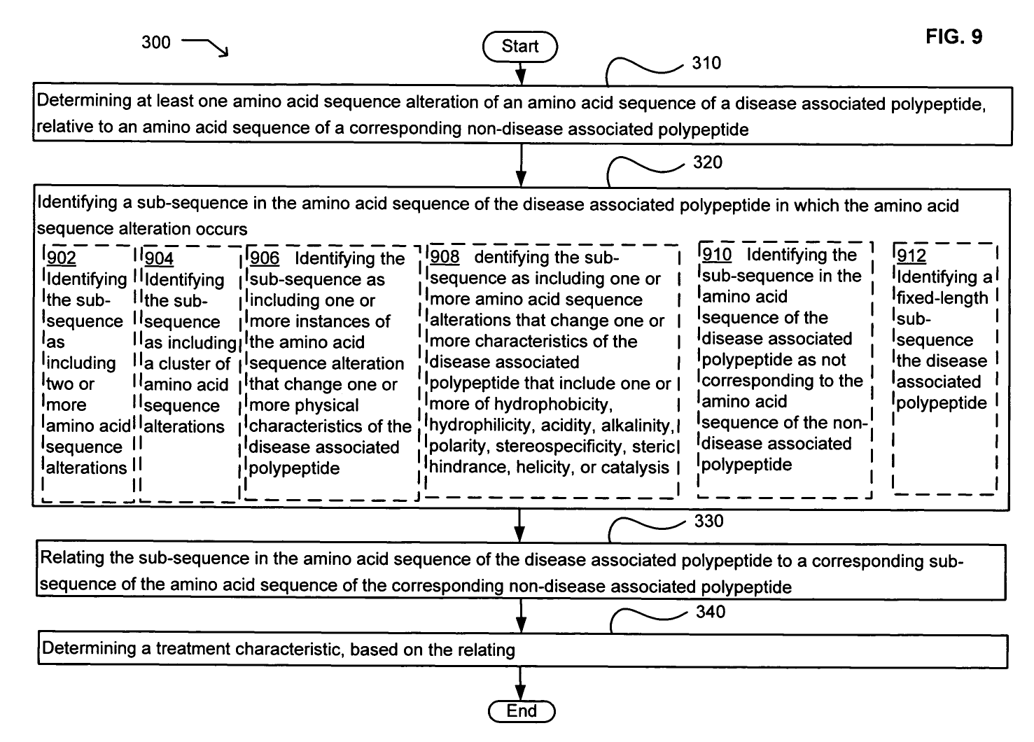

FIG. 9 illustrates alternative embodiments of the example operational flow 300 of FIG. 3. FIG. 9 illustrates example embodiments where the identifying operation 320 may include at least one additional operation. Additional operations may include an operation 902, an operation 904, an operation 906, an operation 909, and operation 910, and/or an operation 912.

At the operation 902, the sub-sequence may be identified as including two or more amino acid sequence alterations. For example, the alteration location analyzer 116 may be configured to identify the sub-sequence 224 or 226, each of which may include two or more amino acid sequence alterations.

At the operation 904, the sub-sequence may be identified as including a cluster of amino acid sequence alterations. For example, the alteration location analyzer 116 may be configured to identify the cluster(s) of amino acid sequence alterations within the sub-sequence 224, which may be associated with a particular motif or other characteristic, as described herein.

At the operation 906, the sub-sequence may be identified as including one or more instances of the amino acid sequence alteration that change one or more physical characteristics of the disease associated polypeptide. For example, the alteration location analyzer 116 may be configured to identify a particular amino acid sequence alteration that affects a physical characteristic of the disease associated polypeptide as a whole, e.g., changes a structure or catalytic activity of the disease associated polypeptide 204.

At the operation 908, the sub-sequence may be identified as including one or more amino acid sequence alterations that change one or more characteristics of the disease associated polypeptide that include one or more of hydrophobicity, hydrophilicity, acidity, alkalinity, polarity, stereospecificity, steric hindrance, helicity, or catalysis. For example, the alteration location analyzer 116 may be configured to identify the sub-sequence 222 or 224, which may change a polarity or catalysis of associated amino acids, respectively.

At the operation 910, the sub-sequence may be identified in the amino acid sequence of the disease associated polypeptide as not corresponding to the amino acid sequence of the non-disease associated polypeptide. For example, the alteration location analyzer 116 may be configured to identify a sub-sequence that resulted from one or more insertions of amino acids.

At the operation 912, a fixed-length sub-sequence the disease associated polypeptide may be identified. For example, the alteration location analyzer 116 may be configured to identify chains of amino acids of a fixed length, e.g., 8 amino acids long, within the disease associated polypeptides 204, 206, 208. Each such chain may be considered to be a quasi or candidate epitope, as described herein, and may be evaluated against corresponding fixed-length chains within the non-disease associated polypeptide 202. As described in more detail herein, the corresponding fixed-length chains within the non-disease associated polypeptide 202 need not be at exactly the same site(s) within the non-disease associated polypeptide, due, for example, to insertions or shifts of amino acids associated with various amino acid sequence alterations.

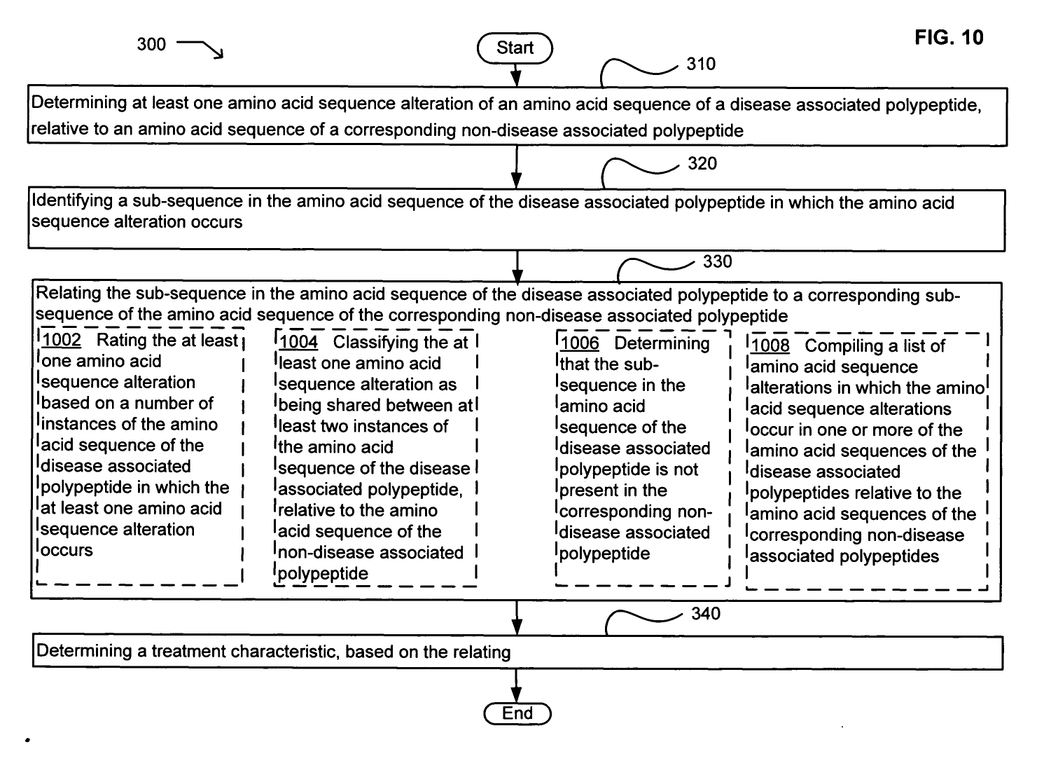

FIG. 10 illustrates alternative embodiments of the example operational flow 300 of FIG. 3. FIG. 10 illustrates example embodiments where the relating operation 330 may include at least one additional operation. Additional operations may include an operation 1002, an operation 1004, an operation 1006, and/or an operation 1008.

At the operation 1002, the at least one amino acid sequence alteration may be rated based on a number of instances of the amino acid sequence of the disease associated polypeptide in which the at least one amino acid sequence alteration occurs. For example, the alteration analyzer 120 may be configured to determine whether the amino acid sequence alterations 210-220 occur in a certain number of patients (e.g., in three patients corresponding to the disease associated polypeptides 204, 206, 208, or in a larger number of patients if available). This information may be treatment-relevant, since, for example, an appearance of a particular amino acid sequence alteration in a large number or percentage of cancer patients may indicate a high degree of correlation between the alteration and the cancer in question, so that ranking the determined alterations based on this criteria may be useful to the treatment logic 122, as described in more detail, below.

At the operation 1004, the at least one amino acid sequence alteration may be classified as being shared between at least two instances of the amino acid sequence of the disease associated polypeptide, relative to the amino acid sequence of the non-disease associated polypeptide. For example, and similar to the example just given, the alteration analyzer 120 may be configured to classify each amino acid sequence alteration 210-220 as occurring in “n” number of patients. The sharing may be a strong, e.g., identical, sharing, where each of two or more patients experience the same mutation at the same site (e.g., where the sub-sequence 222 represents a case where a first amino acid at site 2 of the non-disease associated polypeptide 202 is transformed into a second amino acid at site 2 of all of the disease associated polypeptides 204, 206, 208), or a weaker sharing (e.g., where the sub-sequence 222 may represent a case where a first amino acid at site 2 of the non-disease associated polypeptide 202 is transformed into one of a plurality of amino acids at site 2 of the disease associated polypeptides 204, 206, 208, but where the plurality of amino acids share some common characteristic or property).

At the operation 1006, the sub-sequence in the amino acid sequence of the disease associated polypeptide may be determined not to be present in the corresponding non-disease associated polypeptide. For example, the alteration analyzer 120 may be configured to determine that such a sub-sequence results in whole or in part from an alteration that includes insertions of amino acids into a polymer chain or sequence of amino acids. Such a determination again may be treatment-relevant, since the absence of the sub-sequence may be indicative of a reduced chance that the sub-sequence will stimulate an auto-immune response if administered to the patient 106 (e.g., administration of a sub-sequence that is not naturally or commonly occurring may not stimulate an immune response to such naturally occurring substances).

At the operation 1008, a list of amino acid sequence alterations may be compiled in which the amino acid sequence alterations occur in one or more of the amino acid sequences of the disease associated polypeptides relative to the amino acid sequences of the corresponding non-disease associated polypeptides. For example, the alteration analyzer 120 may be configured to compile such a list as treatment relevant information that may be used by the treatment logic 122 in determining a treatment characteristic, as described herein.

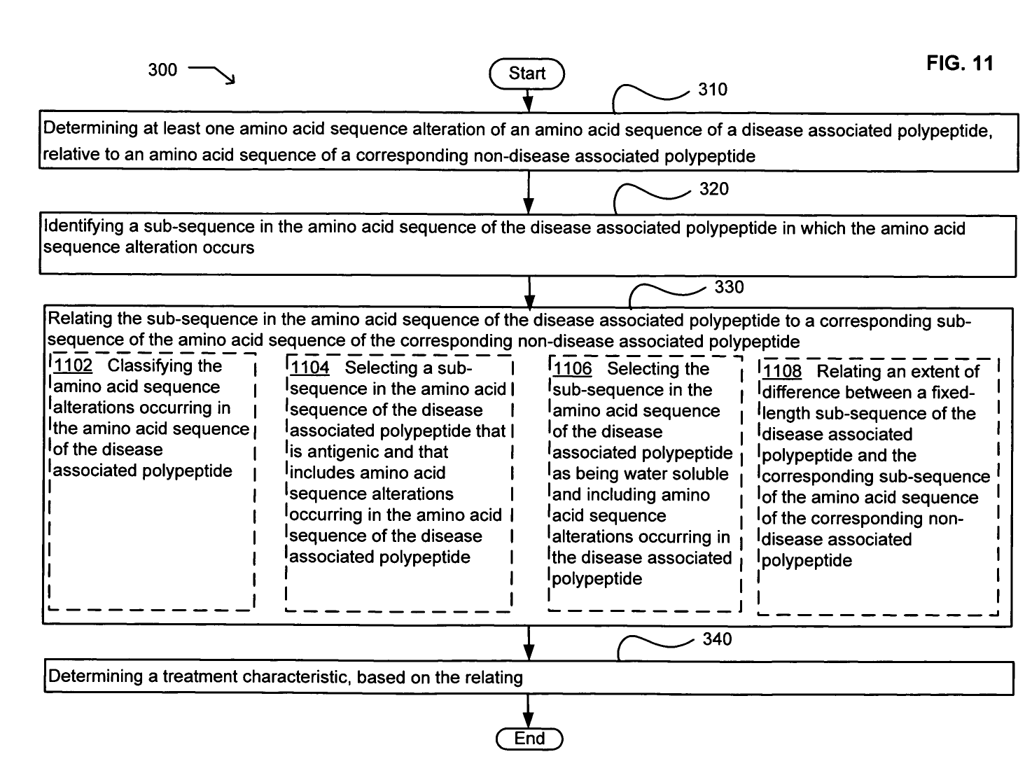

FIG. 11 illustrates alternative embodiments of the example operational flow 300 of FIG. 3. FIG. 11 illustrates example embodiments where the relating operation 330 may include at least one additional operation. Additional operations may include an operation 1002, an operation 1004, an operation 1006, and/or an operation 1008.

At the operation 1102, the amino acid sequence alterations occurring in the amino acid sequence of the disease associated polypeptide may be classified. For example, the alteration analyzer 120 may be configured to classify the amino acid sequence alterations based on physical, chemical, or structural properties, or on frequency of occurrence, or on direct correspondence (or lack thereof) to the non-disease associated polypeptide 202.

At the operation 1104, a sub-sequence in the amino acid sequence of the disease associated polypeptide may be selected that is antigenic and that includes amino acid sequence alterations occurring in the amino acid sequence of the disease associated polypeptide. For example, the alteration analyzer 120 may be configured to determine such antigenic (e.g., immune-stimulating) sub-sequences, perhaps based on known antigenic characteristics of the sub-sequence(s) in question.

At the operation 1106, the sub-sequence in the amino acid sequence of the disease associated polypeptide may be selected as being water soluble and including amino acid sequence alterations occurring in the disease associated polypeptide. For example, the alteration analyzer 120 may be configured to select identified sub-sequence(s) based on the criteria of water-solubility, where such information may be treatment-relevant (for example, such water-soluble sub-sequences may be more suitable for use in preparing and administering a vaccine that includes the sub-sequence).

At the operation 1108, an extent of difference between a fixed-length sub-sequence of the disease associated polypeptide and the corresponding sub-sequence of the amino acid sequence of the corresponding non-disease associated polypeptide may be related. For example, the alteration analyzer 120 may be configured to determine that the fixed-length sub-sequence of the disease associated polypeptide 204 may be longer or shorter (e.g., due to frameshifts or insertions) than a corresponding fixed-length sub-sequence of the non-disease associated polypeptide 202. In other examples, the alteration analyzer 120 may be configured to determine that some number or percentage of amino acids in the fixed-length sub-sequence of the disease associated polypeptide 204 may differ from a corresponding fixed-length sub-sequence of the non-disease associated polypeptide 202 with respect to some characteristic(s), such as those mentioned herein or others, which may include polarity, cysteine/histidine identify, acidity, basicity, aromaticity, aliphaticity, hydrogen-bonding tendencies, or other properties or characteristics. The alteration analyzer 120 may then, for example, assign different values or rankings to different sub-sequences (e.g., may assign a higher ranking to a sub-sequence in which all amino acids have a different length or polarity than a sub-sequence in which only some of the amino acids have a different length or polarity).

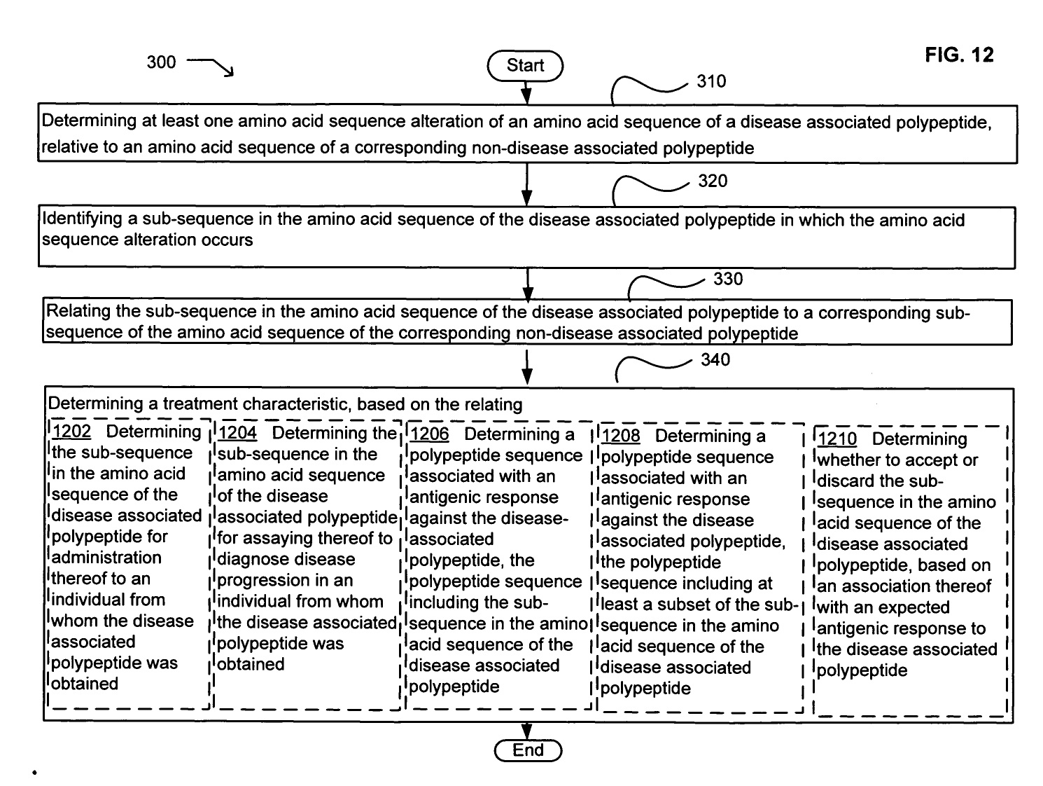

FIG. 12 illustrates alternative embodiments of the example operational flow 300 of FIG. 3. FIG. 12 illustrates example embodiments where the determining operation 340 may include at least one additional operation. Additional operations may include an operation 1202, an operation 1204, an operation 1206, an operation 1208, and/or an operation 1210.

At the operation 1202, the sub-sequence in the amino acid sequence of the disease associated polypeptide may be determined for administration thereof to an individual from whom the disease associated polypeptide was obtained. For example, the treatment logic 122 may be configured to select the sub-sequence 222 for administration to the patient 106, from whom one or more of the non-disease associated polypeptide 202 and/or the disease associated polypeptide(s) 204, 206, 208 may have been obtained.

At the operation 1204, the sub-sequence in the amino acid sequence of the disease associated polypeptide may be determined for assaying thereof to diagnose disease progression in an individual from whom the disease associated polypeptide was obtained. For example, the treatment logic 122 may be configured to assay the sub-sequence 122 to determine properties thereof for diagnosing progression of a cancer in the patient 106.

At the operation 1206, a polypeptide sequence associated with an antigenic response against the disease-associated polypeptide may be determined, the polypeptide sequence including the sub-sequence in the amino acid sequence of the disease associated polypeptide. For example, the treatment logic 122 may be configured to determine that if the sub-sequence 224 has an antigenic property and is desired for treating the patient 106 (e.g., for constructing a vaccine to administer to the patient 106), all of the illustrated disease-associated polypeptide 204 should be used in association with administering the vaccine.

At the operation 1208, a polypeptide sequence associated with an antigenic response against the disease associated polypeptide may be determined, the polypeptide sequence including at least a subset of the sub-sequence in the amino acid sequence of the disease associated polypeptide. For example, the treatment logic 122 may be configured to determine that if the sub-sequence 226 has an antigenic property and is desired for treating the patient 106 (e.g., for constructing a vaccine to administer to the patient 106), then only a subset of the sub-sequence (e.g., the amino acids forming the sub-sequence 224) should be used in association with administering the vaccine.

At the operation 1210, it may be determined whether to accept or discard the sub-sequence in the amino acid sequence of the disease associated polypeptide, based on an association thereof with an expected antigenic response to the disease associated polypeptide. For example, the treatment logic 122 may be configured to determine a ranked level of expected antigenic response of the sub-sequence(s) 222 and 224 (and others), and to filter out one or both of the sub-sequences if their expected antigenic response is below a defined level.

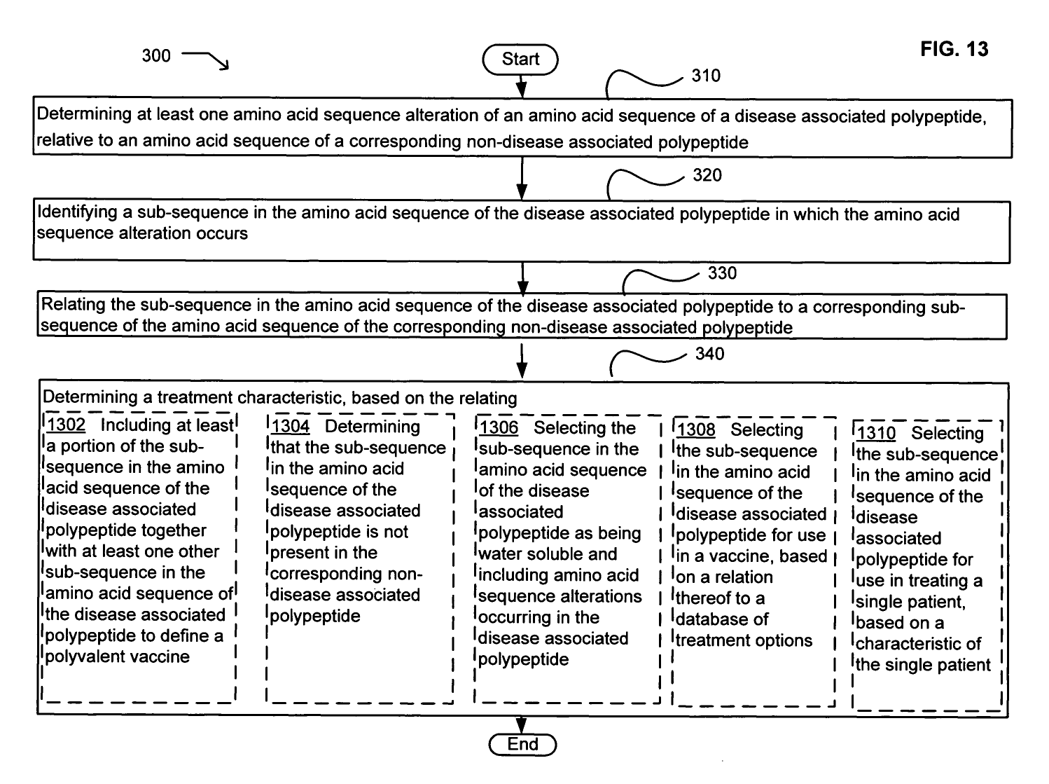

FIG. 13 illustrates alternative embodiments of the example operational flow 300 of FIG. 3. FIG. 13 illustrates example embodiments where the determining operation 330 may include at least one additional operation. Additional operations may include an operation 1302, an operation 1304, an operation 1306, an operation 1308, and/or an operation 1310.

At the operation 1302, at least a portion of the sub-sequence in the amino acid sequence of the disease associated polypeptide may be included together with at least one other sub-sequence in the amino acid sequence of the disease associated polypeptide to define a polyvalent vaccine. For example, the treatment logic 122 may be configured to determine that both the sub-sequence 222 and the sub-sequence 224 are equally valid possibilities for constructing a vaccine, and may combine the two sub-sequences into a polyvalent vaccine accordingly. In other examples, the treatment logic 122 may determine that the sub-sequences 222 and 224 are potentially useful in different ways, due to different classifications thereof. For example, the sub-sequence 222 may be determined to be water-soluble, while the sub-sequence 224 may be determined to have a low likelihood of stimulating an auto-immune response. Consequently, both of the sub-sequences 222, 224 may be used in a polyvalent vaccine, in order to make use of the advantages of both.

At the operation 1304, the sub-sequence in the amino acid sequence of the disease associated polypeptide may be determined not to be present in the corresponding non-disease associated polypeptide. For example, the treatment logic 122 may be configured to determine that a sub-sequence including the amino acid sequence alteration 218 may not be present in the non-disease associated polypeptide 202, so that such a sub-sequence may have a low expectation or danger of stimulating an auto-immune response in the patient 106 (e.g., where the non-disease associated polypeptide 202 was taken from the patient 106).

At the operation 1306, the sub-sequence in the amino acid sequence of the disease associated polypeptide may be selected as being water soluble and including amino acid sequence alterations occurring in the disease associated polypeptide. For example, as referenced herein, the treatment logic 122 may be configured to select the sub-sequence 224 based on expected extent to which the sub-sequence is water soluble.

At the operation 1308, the sub-sequence in the amino acid sequence of the disease associated polypeptide may be selected for use in a vaccine, based on a relation thereof to a database of treatment options. For example, the treatment logic 122 may be configured to access the treatment options database 124 to select the sub-sequence based on information contained therein, as described in more detail herein.

At the operation 1310, the sub-sequence in the amino acid sequence of the disease associated polypeptide may be selected for use in treating a single patient, based on a characteristic of the single patient. For example, and continuing the example above, the treatment logic 122 may be configured to access the treatment options database 124 to determine a characteristic of the patient 106, e.g., that the patient 106 also suffers from an auto-immune disorder and that therefore a premium should be placed on selection of sub-sequences known or expected not to stimulate an auto-immune response.

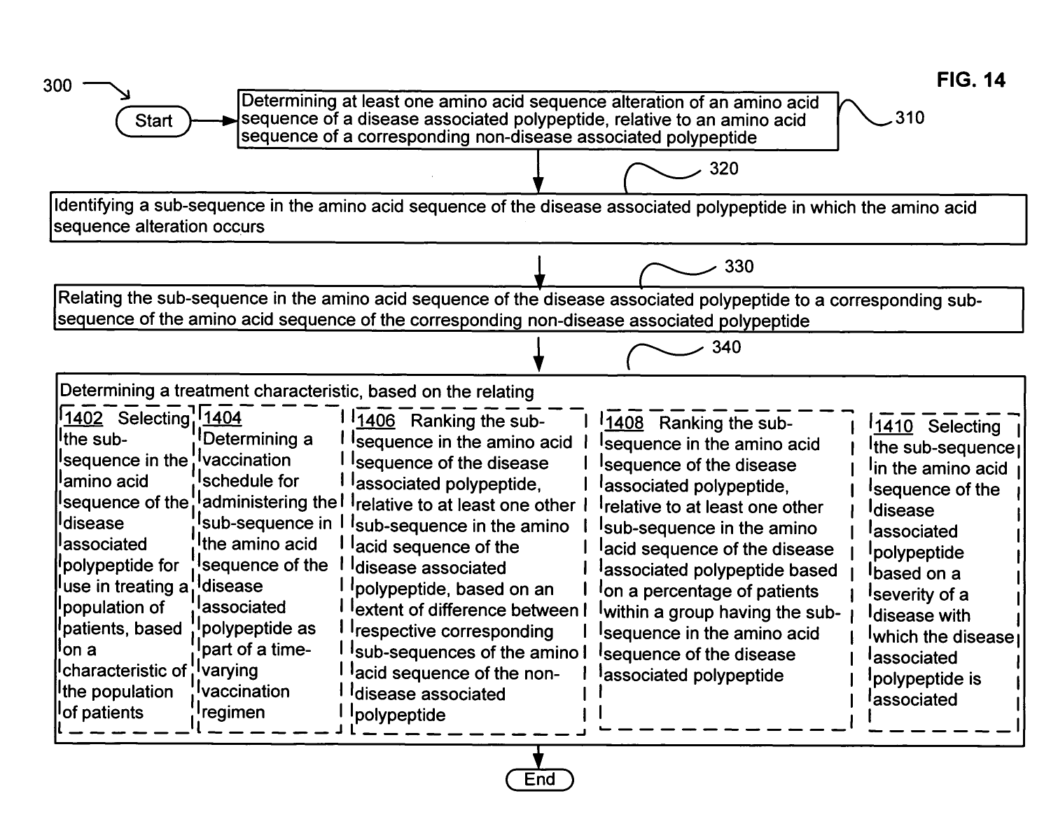

FIG. 14 illustrates alternative embodiments of the example operational flow 300 of FIG. 4. FIG. 14 illustrates example embodiments where the determining operation 340 may include at least one additional operation. Additional operations may include an operation 1402, an operation 1404, an operation 1406, an operation 1408, and/or an operation 1410.

At the operation 1402, the sub-sequence in the amino acid sequence of the disease associated polypeptide may be selected for use in treating a population of patients, based on a characteristic of the population of patients. For example, the treatment logic 122 may be configured to analyze or access characteristics of the patient 106, including, for example, age, gender, ethnicity, or genetic predisposition, and to select the sub-sequence(s) based on membership of the patient 106 in the relevant population(s).

At the operation 1404, a vaccination schedule may be determined for administering the sub-sequence in the amino acid sequence of the disease associated polypeptide as part of a time-varying vaccination regimen. For example, the treatment logic 122 may be configured to determine and output, e.g., by way of the user interface 126, a schedule of administration of a vaccine that includes the sub-sequence in the amino acid sequence of the disease associated polypeptide 204. The schedule may be determined, for example, based on an expected immune response of the patient 106, and may include a series of discrete administrations over a period of weeks, months, or years, or may include one or more continuous administrations, each lasting for a designated amount of time, or some other time-varying administration schedule.

At the operation 1406, the sub-sequence in the amino acid sequence of the disease associated polypeptide may be ranked relative to at least one other sub-sequence in the amino acid sequence of the disease associated polypeptide, based on an extent of difference between respective corresponding sub-sequences of the amino acid sequence of the non-disease associated polypeptide. For example, the treatment logic 122 may be configured to determine that a sub-sequence of the disease associated polypeptide may not appear anywhere in the non-disease associated polypeptide 202. The treatment logic 122 also may determine that the sub-sequence of the disease associated polypeptide may not appear exactly in the corresponding sub-sequence of the non-disease associated polypeptide 202, but may differ to varying extents (e.g., may have a certain number or percentage of amino acids that differ in some defined characteristic, such as a physical, chemical, or structural characteristic.

At the operation 1408, the sub-sequence in the amino acid sequence of the disease associated polypeptide may be ranked relative to at least one other sub-sequence in the amino acid sequence of the disease associated polypeptide based on a percentage of patients within a group having the sub-sequence in the amino acid sequence of the disease associated polypeptide. For example, the treatment logic 122 may be configured to determine that out of a number of patients suffering from a certain type of cancer, all of the patients have the sub-sequence 222, while only some of the patients have the sub-sequence 224, so that the sub-sequence 222 may be considered to be potentially more effective in treating the cancer in question.

At the operation 1410, the sub-sequence in the amino acid sequence of the disease associated polypeptide may be selected based on a severity of a disease with which the disease associated polypeptide is associated. For example, the treatment logic 122 may be configured to filter available sub-sequences based on a safety criteria associated with each, e.g., as determined from the treatment options database 124. In cases of severe illness, this safety criteria may be relaxed in order to utilize a fuller range of possible treatment agents.

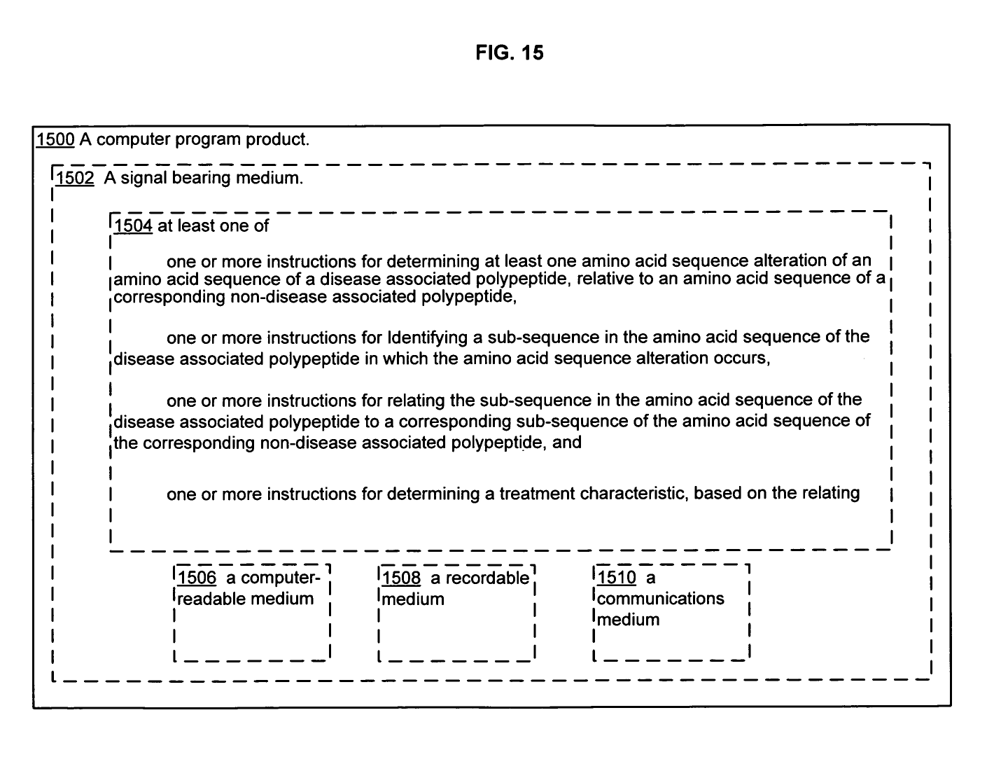

FIG. 15 illustrates a partial view of an example computer program product 1500 that includes a computer program 1504 for executing a computer process on a computing device. An embodiment of the example computer program product 1500 is provided using a signal bearing medium 1502, and may include one or more instructions for determining at least one amino acid sequence alteration of an amino acid sequence of a disease associated polypeptide, relative to an amino acid sequence of a corresponding non-disease associated polypeptide. The signal bearing medium also may bear one or more instructions for identifying a sub-sequence in the amino acid sequence of the disease associated polypeptide in which the amino acid sequence alteration occurs. The signal bearing medium also may bear one or more instructions for relating the sub-sequence in the amino acid sequence of the disease associated polypeptide to a corresponding sub-sequence of the amino acid sequence of the corresponding non-disease associated polypeptide. The signal bearing medium also may bear one or more instructions for determining a treatment characteristic, based on the relating.

The one or more instructions may be, for example, computer executable and/or logic-implemented instructions. In one implementation, the signal-bearing medium 1502 may include a computer-readable medium 1506. In one implementation, the signal bearing medium 1502 may include a recordable medium 1508. In one implementation, the signal bearing medium 1502 may include a communications medium 1510.

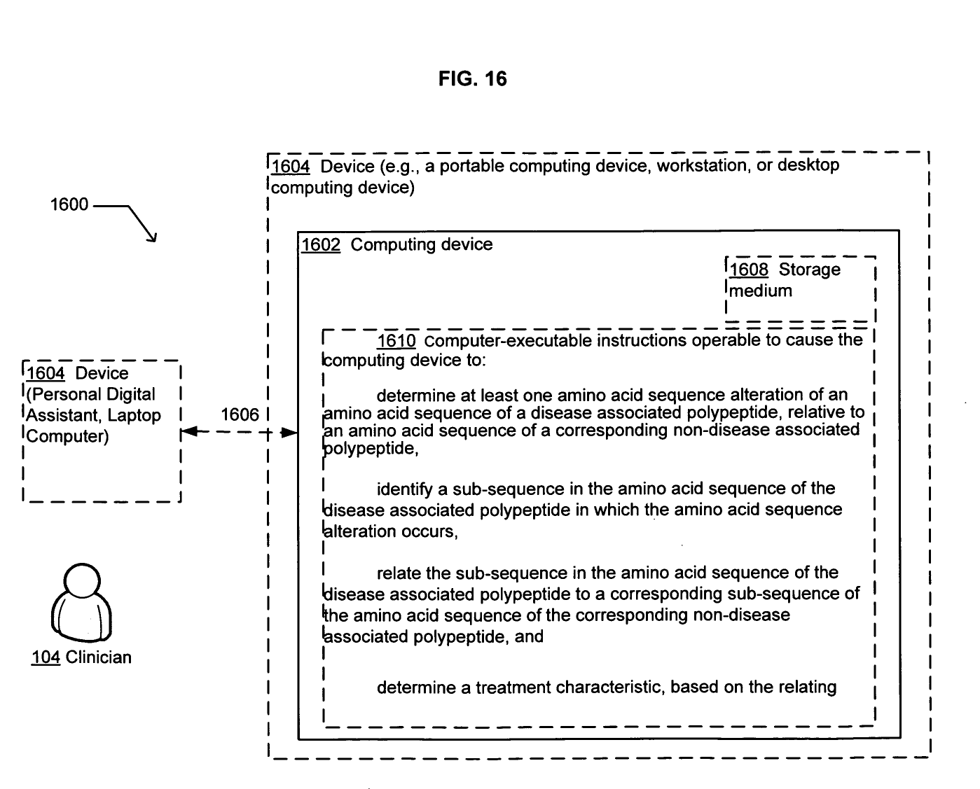

FIG. 16 illustrates an example system 1600 in which embodiments may be implemented. The system 1600 includes a computing system environment. The system 1600 also illustrates the clinician 104 using a device 1604, which is optionally shown as being in communication with a computing device 1602 by way of an optional coupling 1606. The optional coupling 1606 may represent a local, wide-area, or peer-to-peer network, or may represent a bus that is internal to a computing device (e.g., in example embodiments in which the computing device 1602 is contained in whole or in part within the device 1604). A storage medium 1608 may be any computer storage media.

The computing device 1602 includes computer-executable instructions 1610 that when executed on the computing device 1602, cause the computing device 1602 to determine at least one amino acid sequence alteration of an amino acid sequence of a disease associated polypeptide, relative to an amino acid sequence of a corresponding non-disease associated polypeptide, identify a sub-sequence in the amino acid sequence of the disease associated polypeptide in which the amino acid sequence alteration occurs, relate the sub-sequence in the amino acid sequence of the disease associated polypeptide to a corresponding sub-sequence of the amino acid sequence of the corresponding non-disease associated polypeptide, and determine a treatment characteristic, based on the relating.

In FIG. 16, then, the system 1600 includes at least one computing device (e.g., 1602 and/or 1604). The computer-executable instructions 1610 may be executed on one or more of the at least one computing device. For example, the computing device 1602 may implement the computer-executable instructions 1610 and output a result to (and/or receive data from) the computing (clinician) device 1604. Since the computing device 1602 may be wholly or partially contained within the computing (clinician) device 1604, the computing (clinician) device 1604 also may be said to execute some or all of the computer-executable instructions 1610, in order to be caused to perform or implement, for example, various ones of the techniques described herein, or other techniques.

The clinician device 1604 may include, for example, one or more of a personal digital assistant (PDA), a laptop computer, a tablet personal computer, a networked computer, a computing system comprised of a cluster of processors, a workstation computer, and/or a desktop computer. In another example embodiment, the clinician device 1604 may be operable to communicate with the computing device 1602 to communicate with a database (e.g., implemented using the storage medium 1608) to access the at least one treatment parameter(s).

The following reference(s) are hereby incorporated by reference in its entirety to the extent such is not inconsistent herewith:

Sjöblom, et al., “The Consensus Coding Sequences of Human Breast and Colorectal Cancers,” Science 314, pages 268-274, published online 6 Sep. 2006 [DOI: 10.1126/science.1133427]

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality. Any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

While particular aspects of the present subject matter described herein have been shown and described, it will be apparent to those skilled in the art that, based upon the teachings herein, changes and modifications may be made without departing from this subject matter described herein and its broader aspects and, therefore, the appended claims are to encompass within their scope all such changes and modifications as are within the true spirit and scope of this subject matter described herein. Furthermore, it is to be understood that the invention is solely defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

1. A method comprising:
determining at least one amino acid sequence alteration of an amino acid sequence of a disease associated polypeptide, relative to an amino acid sequence of a corresponding non-disease associated polypeptide;
identifying a sub-sequence in the amino acid sequence of the disease associated polypeptide in which the amino acid sequence alteration occurs;
relating the sub-sequence in the amino acid sequence of the disease associated polypeptide to a corresponding sub-sequence of the amino acid sequence of the corresponding non-disease associated polypeptide; and
determining a treatment characteristic, based on the relating.

2. (canceled)

3. The method of claim 1, wherein determining at least one amino acid sequence alteration of an amino acid sequence of a disease associated polypeptide, relative to an amino acid sequence of a corresponding non-disease associated polypeptide comprises:
accessing an amino acid sequence corresponding to the disease associated polypeptide from a memory that includes a database of cancer associated polypeptides.

4.-5. (canceled)

6. The method of claim 1, wherein determining at least one amino acid sequence alteration of an amino acid sequence of a disease associated polypeptide, relative to an amino acid sequence of a corresponding non-disease associated polypeptide comprises:
accessing an amino acid sequence corresponding to the non-disease associated polypeptide from a memory that includes a database of non-disease associated polypeptides.

7. The method of claim 1, wherein determining at least one amino acid sequence alteration of an amino acid sequence of a disease associated polypeptide, relative to an amino acid sequence of a corresponding non-disease associated polypeptide comprises:
determining at least one amino acid sequence alteration of a disease associated polypeptide relative to an amino acid sequence of a corresponding non-disease associated polypeptide within a single individual.

8. The method of claim 1, wherein determining at least one amino acid sequence alteration of an amino acid sequence of a disease associated polypeptide, relative to an amino acid sequence of a corresponding non-disease associated polypeptide comprises:
determining at least one amino acid sequence alteration of a disease associated polypeptide from a first individual relative to an amino acid sequence of a corresponding non-disease associated polypeptide from a second individual.

9.-19. (canceled)

20. The method of claim 1, wherein identifying a sub-sequence in the amino acid sequence of the disease associated polypeptide in which the amino acid sequence alteration occurs comprises:
identifying a range of amino acid positions within the sub-sequence within which the amino acid sequence alteration occurs in at least one other instance of the amino acid sequence of the disease associated polypeptide.

21. The method of claim 1, wherein identifying a sub-sequence in the amino acid sequence of the disease associated polypeptide in which the amino acid sequence alteration occurs comprises:
performing pattern recognition and/or statistical analysis of a plurality of instances of the amino acid sequence of the disease associated polypeptide, relative to one another.

22. (canceled)

23. The method of claim 1, wherein identifying a sub-sequence in the amino acid sequence of the disease associated polypeptide in which the amino acid sequence alteration occurs comprises:
identifying the sub-sequence as including at least one amino acid motif.

24. (canceled)

25. The method of claim 1, wherein identifying a sub-sequence in the amino acid sequence of the disease associated polypeptide in which the amino acid sequence alteration occurs comprises:
identifying the sub-sequence as including a cluster of amino acid sequence alterations.

26.-27. (canceled)

28. The method of claim 1I wherein identifying a sub-sequence in the amino acid sequence of the disease associated polypeptide in which the amino acid sequence alteration occurs comprises:
identifying the sub-sequence in the amino acid sequence of the disease associated polypeptide as not corresponding to the amino acid sequence of the non-disease associated polypeptide.

29.-31. (canceled)

32. The method of claim 1, wherein relating the sub-sequence in the amino acid sequence of the disease associated polypeptide to a corresponding sub-sequence of the amino acid sequence of the corresponding non-disease associated polypeptide comprises:
determining that the sub-sequence in the amino acid sequence of the disease associated polypeptide is not present in the corresponding non-disease associated polypeptide.

33.-34. (canceled)

35. The method of claim 1, wherein relating the sub-sequence in the amino acid sequence of the disease associated polypeptide to a corresponding sub-sequence of the amino acid sequence of the corresponding non-disease associated polypeptide comprises:
selecting a sub-sequence in the amino acid sequence of the disease associated polypeptide that is antigenic and that includes amino acid sequence alterations occurring in the amino acid sequence of the disease associated polypeptide.

36.-37. (canceled)

38. The method of claim 1, wherein determining a treatment characteristic, based on the relating comprises:
determining the sub-sequence in the amino acid sequence of the disease associated polypeptide for administration thereof to an individual from whom the disease associated polypeptide was obtained.

39. The method of claim 1, wherein determining a treatment characteristic, based on the relating comprises:
determining the sub-sequence in the amino acid sequence of the disease associated polypeptide for assaying thereof to diagnose disease progression in an individual from whom the disease associated polypeptide was obtained.

40.-42. (canceled)

43. The method of claim 1, wherein determining a treatment characteristic, based on the relating comprises:
including at least a portion of the sub-sequence in the amino acid sequence of the disease associated polypeptide together with at least one other sub-sequence in the amino acid sequence of the disease associated polypeptide to define a polyvalent vaccine.

44. The method of claim 1, wherein determining a treatment characteristic, based on the relating comprises:
determining that the sub-sequence in the amino acid sequence of the disease associated polypeptide is not present in the corresponding non-disease associated polypeptide.

45.-48. (canceled)

49. The method of claim 1, wherein determining a treatment characteristic, based on the relating comprises:
determining a vaccination schedule for administering the sub-sequence in the amino acid sequence of the disease associated polypeptide as part of a time-varying vaccination regimen.

50.-51. (canceled)

52. The method of claim 1, wherein determining a treatment characteristic, based on the relating comprises:
selecting the sub-sequence in the amino acid sequence of the disease associated polypeptide based on a severity of a disease with which the disease associated polypeptide is associated.

53. A computer program product comprising:
a signal-bearing medium bearing
(a) one or more instructions for determining at least one amino acid sequence alteration of an amino acid sequence of a disease associated polypeptide, relative to an amino acid sequence of a corresponding non-disease associated polypeptide,
(b) one or more instructions for Identifying a sub-sequence in the amino acid sequence of the disease associated polypeptide in which the amino acid sequence alteration occurs,
(c) one or more instructions for relating the sub-sequence in the amino acid sequence of the disease associated polypeptide to a corresponding sub-sequence of the amino acid sequence of the corresponding non-disease associated polypeptide, and
(d) one or more instructions for determining a treatment characteristic, based on the relating.

54. The computer program product of claim 53, wherein the signal-bearing medium includes a computer-readable medium.

55. The computer program product of claim 53, wherein the signal-bearing medium includes a recordable medium.

56. The computer program product of claim 53, wherein the signal-bearing medium includes a communications medium.

57. A system comprising:
means for determining at least one amino acid sequence alteration of an amino acid sequence of a disease associated polypeptide, relative to an amino acid sequence of a corresponding non-disease associated polypeptide;
means for identifying a sub-sequence in the amino acid sequence of the disease associated polypeptide in which the amino acid sequence alteration occurs;
means for relating the sub-sequence in the amino acid sequence of the disease associated polypeptide to a corresponding sub-sequence of the amino acid sequence of the corresponding non-disease associated polypeptide; and
means for determining a treatment characteristic, based on the relating.

58.-108. (canceled)

109. A system comprising:
a computing device including computer-executable instructions that when executed on the computing device, cause the computing device to
determine at least one amino acid sequence alteration of an amino acid sequence of a disease associated polypeptide, relative to an amino acid sequence of a corresponding non-disease associated polypeptide,
identify a sub-sequence in the amino acid sequence of the disease associated polypeptide in which the amino acid sequence alteration occurs,
relate the sub-sequence in the amino acid sequence of the disease associated polypeptide to a corresponding sub-sequence of the amino acid sequence of the corresponding non-disease associated polypeptide, and
determine a treatment characteristic, based on the relating.

110. (canceled)

111. A treatment system, the treatment system comprising
(a) alteration determination logic configured to determine at least one amino acid sequence alteration of an amino acid sequence of a disease associated polypeptide, relative to an amino acid sequence of a corresponding non-disease associated polypeptide,
(b) alteration location logic configured to identify a sub-sequence in the amino acid sequence of the disease associated polypeptide in which the amino acid sequence alteration occurs,
(c) alteration analyzer configured to relate the sub-sequence in the amino acid sequence of the disease associated polypeptide to a corresponding sub-sequence of the amino acid sequence of the corresponding non-disease associated polypeptide, and
(d) treatment logic configured to determine a treatment characteristic, based on the relating.
